(19) United States
(12) Patent Application Publication
(10) Pub. No.: US 2021/0353781 A1
(43) Pub. Date: Nov. 18, 2021

(54) SURFACE-MODIFIED CELLS, METHODS OF MAKING AND USING

(71) Applicants: Massachusetts Institute of Technology, Cambridge, MA (US); The Children's Medical Center Corporation, Boston, MA (US)

(72) Inventors: Omid Veiseh, Bellaire, TX (US); Volkan Yesilyurt, Watertown, MA (US); Andrew Bader, Charlestown, MA (US); Whitney Loo, Berkeley, CA (US); Daniel G. Anderson, Framingham, MA (US); Robert S. Langer, Newton, MA (US)

(21) Appl. No.: 17/278,383

(22) PCT Filed: Sep. 23, 2019
(86) PCT No.: PCT/US2019/052432

§ 371 Date: Mar. 22, 2021

Related U.S. Application Data
(60) Provisional application No. 62/734,786, filed on Sep. 21, 2018.

(51) Int. Cl. A61K 49/18; A61K 47/69

(52) U.S. Cl. CPC A61K 49/1854; A61K 47/6933; A61K 49/1878; A61K 49/1896

(57) ABSTRACT

Surface-modified cell containing a cell and a conformal coating on the extracellular surface of the cell are described. The conformal coating contains two or more layers containing particles (e.g. nanoparticles) or macromolecules. The cell is an islet cell, a B cell, or a T cell. The macromolecules or particles are formed from zwitterionic polymers. Covalent linkages are employed to link the particles or macromolecules to a cell surface molecule containing an abiotic functional group, or between macromolecules and/or particles in adjacent layers. Also described are methods of making and using a surface-modified cell.

### CROSS-REFERENCE TO RELATED APPLICATION

This application claims the benefit of and priority to U.S. Provisional Application No. 62/734,786 filed Sep. 21, 2018, which is hereby incorporated by reference in its entirety.

### STATEMENT REGARDING FEDERALLY SPONSORED RESEARCH

This invention was made with Government support under Grant No. W81XWH-13-1-0215 awarded by the Department of Defense. The Government has certain rights in the invention.

### FIELD OF THE INVENTION

The invention is generally in the field of cell surface modification, particularly chemical modification of cells via a conformal coating with macromolecules and/or particles (such as nanoparticles) for cell-based therapies.

### BACKGROUND OF THE INVENTION

Cell-based therapies are rapidly emerging as new approaches for the treatment of a number of diseases, including cancer, diabetes, obesity and heart disease. Examples of approaches describing experimentation using cell-based therapies include the administration of: engineered T cells to treat cancer (Davilla, et al., Sci. Transl. Med. 2014, 6(224), 224ra225; Stromnes, et al., Immunol. Rev. 2014, 257(1), 145-164), pancreatic islet cells to treat type-1 diabetes (Shapiro, et al., N. Engl. J. Med. 2006, 355(13), 1318-1330), cardiomyocytes to treat cardiovascular diseases (Ishigami, et al., Circ. Res. 2015, 116(4), 653-664), and brown fat adipose tissue to treat obesity (Liu, et al., Endocrinology 2015, en20141598).

Many cell-based therapies involve modifying the surface of a cell prior to administering to a host (Bratlie, et al., Adv. Healthc. Mater. 2012, 1(3), 267-284; Stephan, et al., Nano Today 2011, 6(3), 309-325; Zhang, et al., Polymers 2017, 9, 40). Cell surface modification can lead to methods of tissue targeting (Saxon and Bertozzi, Science 2000, 287, 2007-2010); enhancing cell longevity; altering cell-cell and cell-extracellular interactions (Stephan, et al., Nano Today 2011, 6(3)); and isolating cells from the immune system of a host (Bratlie, et al., Adv. Healthc. Mater. 2012, 1(3), 267-284).

However, several problems remain in modifying the surface of a cell for effective application in clinically-relevant settings. For instance, cells displaying exogenous materials on their surfaces usually induce a foreign body response from a host's immune system (Zhang, et al., Polymers 2017, 9, 40; Bratlie, et al., Adv. Healthc. Mater. 2012, 1(3), 267-284). The final pathological product of this response is fibrosis, which is characterized by the accumulation of excessive extracellular matrix around the administered exogenous material and is a key obstacle for implantable exogenous materials as the matrix isolates the material, and consequently the cell, from the host (Wick, et al., Annu. Rev. Immunol. 2013, 31:107-135; Wynn & Ramalingam, Nat. Med. 2012, 18:1028-1040). The fibrous tissue surrounding the exogenous material reduces the diffusion of nutrients and oxygen to the cells, causing them to die. In addition, the cell membrane is highly dynamic and continuously redistributes cell surface molecules on the membrane and/or rapidly internalizes these molecules (Stephan, et al., Nano Today 2011; Zhang, et al., Polymers 2017, 9, 40). Further, interactions between positive charges in a material used to modify a cell's surface and the negative charges naturally present on the surface of a cell, can lead to local membrane depolarization and subsequent internalization of the material (Stephan, et al., Nano Today 2011). Redistribution or internalization of cell surface molecules can trigger a similar outcome to materials used to modify a cell's surface, which results in the loss or degradation of these materials or their functions on the cell's surface.

Accordingly, the development of cells containing stably modified surfaces, cells with modified surfaces that resist host foreign body responses, or both, remains an unmet need, and is an area of active research.

Therefore, it is an object of the invention to provide surface-modified cells with improved properties.

It is also an object of the invention to provide surface-modified cells containing stably modified extracellular surfaces.

It is a further object of the invention to provide methods of making surface-modified cells containing stably modified extracellular surfaces.

### SUMMARY OF THE INVENTION

A surface-modified cell containing a cell and a conformal coating on the extracellular surface of the cell has been developed. The cell is an islet cell, a B cell, or a T cell. The conformal coating contains two or more layers containing particles (e.g. nanoparticles) or macromolecules. Preferably, the particles are nanoparticles. The conformal coating is more stable compared to a coating that contains only a single layer. Further, the number of layers in the conformal coating can be altered to control residence time of cells.

The macromolecules or nanoparticles are formed from zwitterionic polymers (such as zwitterionic polymers that contain a sulfobetaine moiety, a carboxybetaine moiety, or a phosphoryl choline moiety). The macromolecules or nanoparticles in the layer adjacent to the surface of the cell are covalently linked to a cell surface glycoprotein that contains an abiotic functional group. The macromolecules or nanoparticles in a given layer are also covalently linked to the macromolecules or nanoparticles in an adjacent layer. The covalent linkages contain a substituted triazole group.

Optionally, the conformal coating contains a therapeutic, diagnostic, prophylactic, and/or targeting agent.

Methods of making a surface-modified cell involve exploiting the biosynthetic machinery of a cell to introduce an abiotic functional group into a biomolecule that is subsequently expressed on the extracellular surface of the cell. The abiotic functional group is then used to covalently link nanoparticles or macromolecules to the surface of the cell, thereby forming a conformal coating.

### BRIEF DESCRIPTION OF THE DRAWINGS

FIGS. 1A-1C are schematics showing cell surface modification and the formation of a particle (e.g. a nanoparticle, such as a nanogel). FIG. 1A shows how cells can be modified to express chemical functional groups, for further modification, on their surfaces through treatment with a compound containing an abiotic functional group, e.g., an unnatural sugar. FIG. 1B shows the formation of nanoparticles (e.g. a zwitterionic nanogels) from polymers (e.g. zwitterionic polymers). The particles contain an abiotic functional group (e.g. azide) displayed on their surfaces. FIG. 1C shows coating of cells (e.g. pancreatic islets) using nanoparticles such as those formed in FIG. 1B.

### DETAILED DESCRIPTION OF THE INVENTION

### I. Definitions

“Abiotic,” as relates to chemical functional groups, refers to a functional group that is absent from the extracellular surface of a native plasma membrane of a cell.

The terms “biocompatible” as used herein refers to one or more materials that are neither themselves toxic to the host (e.g., an animal or human), nor degrade (if the polymer degrades) at a rate that produces monomeric or oligomeric subunits or other byproducts at toxic concentrations in the host. The term “biodegradable” as used herein means that the materials degrades or breaks down into its component subunits, or digestion, e.g., by a biochemical process, of the polymer into smaller, non-polymeric subunits.

“Biocompatible” refers to a substance or object that performs its desired function when introduced into an organism without inducing significant inflammatory response, immunogenicity, or cytotoxicity to native cells, tissues, or organs. For example, a biocompatible conformal coating is a coating that performs its desired function when introduced into an organism without inducing significant inflammatory response, immunogenicity, or cytotoxicity to native cells, tissues, or organs. Biocompatibility can be quantified using the in vivo biocompatibility assay described below.

In this assay, a substance or object can be considered biocompatible if it produces, in a test of biocompatibility related to immune system reaction, less than 50%, 45%, 40%, 35%, 30%, 25%, 20%, 15%, 10%, 8%, 6%, 5%, 4%, 3%, 2%, or 1% of the reaction, in the same test of biocompatibility, produced by a substance or object the same as the test substance or object except for a lack of the structure or property on the test substance or object. Examples of useful biocompatibility tests include measuring and assessing cytotoxicity in cell culture, inflammatory response after implantation (such as by fluorescence detection of cathepsin activity), and immune system cells recruited to implant (for example, macrophages and neutrophils).

“Building block,” as relates to a biomolecule, refers to a smaller component, e.g. monomer or monomeric unit, of a biomolecule. A biomolecule can be a protein, glycoprotein, lipid, glycolipid, or polynucleotide. A building block can be an amino acid, monosaccharide, fatty acid, or nucleotide. The building block can be a precursor to another building block. For example, 2-deoxyacetylmannosamine is a precursor to N-acetylneuraminic acid (sialic acid) in the biosynthesis of cell surface glycans, glycoproteins, or glycolipids.

“Cell” refers to individual cells, cell lines, primary cultures, or cultures derived from such cells unless specifically indicated. “Culture,” as used herein, refers to a composition including isolated cells of the same or a different type. Preferably, the term “cell” encompasses eukaryotic cells.

“Chemical modification” and related terms, in the context of a cell, refers to chemical modification of the cell. Generally, such chemical modification is by direct attachment, coupling, or adherence of a macromolecule, particle, or both, to the surface of the cell. Preferably, the chemical modification involves modification with one or more of the macromolecules or particles described herein. Chemical modification, in the context of the cell, can be accomplished at any time and in any manner, including, for example, taking advantage of the biosynthetic machinery of the cell to first introduce abiotic functional groups to a biomolecule within a cell, which is eventually expressed on the extracellular surface of the cell. The terms “replaced,” “replace,” “modified,” “chemically modified,” “surface modified,” “modification,” “chemical modification,” “surface modification,” “substituted,” “substitution,” “derived from,” “based on,” or “derivatized,” and similar terms, as used herein to describe a structure, do not limit the structure to one made from a specific starting material or by a particular synthetic route. Except where specifically and expressly provided to the contrary, the terms refer to a structural property, regardless of how the structure was formed, and the structure is not limited to a structure made by any specific method.

“Coating” refers to any temporary, semi-permanent or permanent layer, covering or surface. A coating can be applied as a solution, gas, vapor, liquid, paste, or gel. Elasticity can be engineered into coatings to accommodate pliability, e.g. librations of the cell membrane.

“Conformal,” as relates to a coating, generally means that the features of the object being coated, such as angles, scale, etc. are preserved.

“Conjugate,” and its related terms, refers to the covalent or non-covalent linkage of a molecule to another molecule, or one part of a molecule to a different part of the same molecule. Covalent linkages can be direct or indirect (i.e., mediated via a linker). Non-covalent linkage includes electrostatic interactions, hydrogen bonding interactions, affinity interactions, metal coordination, physical adsorption, host-guest interactions, hydrophobic interactions, n-stacking interactions, van der Waals interactions, magnetic interactions, and dipole-dipole interactions.

“Covalent linkage” refers to a bond or organic moiety that covalently links particles, macromolecules, or both, in a layer adjacent to the surface of a cell to a molecule on the cell's surface. “Covalent linkage” can also refer to a bond or organic moiety that covalently links particles, macromolecules, or both, in one layer to particles, macromolecules, or both, in an adjacent layer in the conformal coating on the surface of a cell.

“Effective amount” and “therapeutically effective amount,” are used interchangeably, and as applied to the surface-modified cell, nanoparticles, therapeutic agents, and compositions described herein, mean the quantity necessary to render the desired therapeutic result. For example, an effective amount is an amount effective to treat, cure, or alleviate the symptoms of a disease for which the composition and/or therapeutic agent, or pharmaceutical composition, is/are being administered. Amounts effective for the particular therapeutic goal sought will depend upon a variety of factors including the disease being treated and its severity and/or stage of development/progression; the bioavailability and activity of the specific compound and/or antineoplastic, or pharmaceutical composition, used; the route or method of administration and introduction site on the subject; the rate of clearance of the specific composition and other pharmacokinetic properties; the duration of treatment; inoculation regimen; drugs used in combination or coincident with the specific composition; the age, body weight, sex, diet, physiology and general health of the subject being treated; and like factors well known to one of skill in the relevant scientific art. Some variation in dosage will necessarily occur depending upon the condition of the subject being treated, and the physician or other individual administering treatment will, in any event, determine the appropriate dosage for an individual patient.

“Foreign body response” as used herein, refers to the immunological response of biological tissue to the presence of any foreign material in the tissue which can include protein adsorption, macrophages, multinucleated foreign body giant cells, fibroblasts, and angiogenesis.

“Glycan” generally refers to monosaccharides, disaccharides, oligosaccharides, or polysaccharides.

“Islet cell” refers to an endocrine cell derived from a mammalian pancreas. Islet cells include alpha cells that secrete glucagon, beta cells that secrete insulin and amylin, delta cells that secrete somatostatin, PP cells that secrete pancreatic polypeptide, or epsilon cells that secrete ghrelin. The term includes homogenous and heterogenous populations of these cells. In preferred embodiments, a population of islet cells contains at least beta cells.

The terms “inhibit” and “reduce” means to reduce or decrease in activity or expression. This can be a complete inhibition or reduction of activity or expression, or a partial inhibition or reduction. Inhibition or reduction can be compared to a control or to a standard level. Inhibition can be 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64,65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100%.

“Particle,” as used herein refers to an entity having a diameter of no more than 10 μm, and includes microparticles and nanoparticles. Microparticles preferably have an average diameter between 1 μm and 10 um, inclusive, more preferably between 1 μm and 5 μm, inclusive. “Nanoparticle” generally refers to a particle having an average diameter, greater than or equal to 1 nm and less than 1 micron. The particles can have any shape. Microparticles and nanoparticles having a spherical shape are generally referred to as “microspheres” and “nanospheres,” respectively.

“Small molecule” generally refers to an organic molecule that is less than about 2000 g/mol in molecular weight, less than about 1500 g/mol, less than about 1000 g/mol, less than about 800 g/mol, or less than about 500 g/mol. In some forms, small molecules are non-polymeric and/or non-oligomeric.

“Stable,” as relates to a conformal coating, refers to percentage of cell-surface coating depreciation over time. Coating coverage (percent of cell surface area covered with coating) can be tuned for each application. In vivo stability can be measured using imaging methods, such as confocal fluorescence imaging and fluorescence lifetime imaging. In vitro stability can be measured using an analytical method such as atomic force microscopy, which give a readout of the thickness of a coating.

“Surface modification” and related terms, in the context of a cell, e.g., refers to chemical modification of the surface or a surface of the cell. Generally, such surface modification is by direct attachment, coupling, or adherence of a compound to the surface material of the product. Preferably, the chemical modification involves modification with one or more of the macromolecules or particles described herein. Chemical modification, in the context of the cell, can be accomplished at any time and in any manner, including, for example, taking advantage of the biosynthetic machinery of the cell to first introduce an abiotic functional group to a biomolecule within a cell, which is eventually expressed on the extracellular surface of the cell. Except where specifically and expressly provided to the contrary, the term “surface modification” refers to a structural property, regardless of how the structure was formed, and the structure is not limited to a structure made by any specific method.

“Transplant” or “administration,” in the context of a surface-modified cell, refers to the transfer of a cell, tissue, or organ to a subject from another source. The term is not limited to a particular mode of transfer. Surface-modified cells may be transplanted by any suitable method, such as by injection or surgical implantation.

“Zwitterion,” “zwitterionic,” and “zwitterionic monomer” are used interchangeably to refer to chemical compound, or a monomer or monomeric unit within a polymer, which contains one or more cationic groups and one or more anionic groups. Typically, the charges on the cationic and anionic groups are balanced, resulting in a monomer with zero net charge. However, it is not necessary that the charges on the cationic and anionic groups balance out.

“Zwitterionic polymer” refers to a polymer that contains at least a zwitterionic monomer, monomers with cationic and anionic groups on different monomer units, or a combination thereof. The zwitterionic polymers can be random copolymers, block copolymers, or a combination thereof.

### II. Surface-Modified Cells

A surface-modified cell containing a cell and a conformal coating on the extracellular surface of the cell. The conformal coating contains one or more layers containing particles (such as microparticles and/or nanoparticles), macromolecules, or both.

Preferably, the conformal coating contains two or more layers containing macromolecules, nanoparticles, or both. It has been discovered that conformal coatings containing two layers formed more stable coatings on the surface of a cell, compared to coatings containing a single layer. It has also been discovered that the residence time of cells in a host can be controlled by changing the number of layers in a conformal coating.

The macromolecules, particles (e.g. nanoparticles), or both, can include a polymer with a backbone formed from synthetic polymers; biopolymers such as carbohydrates, polypeptides, and polynucleotides; blends, and copolymers thereof. The layer of the conformal coating adjacent to the surface of the cell is conjugated to a molecule expressed on the extracellular surface of the cell. Further, adjacent layers within the conformal coating are conjugated to each other.

Optionally, the surface-modified cell contains therapeutic, diagnostic, prophylactic, and/or targeting agents. Preferably, these agents are conjugated to the conformal coating.

Preferably, the cell is an islet cell, a B cell, or a T cell. Preferably macromolecules, particles (e.g. nanoparticles), or both, in the layer adjacent to the surface of the cell are covalently linked to a cell surface molecule. Preferably, the cell surface molecule is a glycoprotein that is modified to contain an abiotic functional group involved in forming the covalent linkage. Preferably, macromolecules, particles (e.g. nanoparticles), or both, in a given layer of the conformal coating are covalently linked to macromolecules, nanoparticles, or both, in an adjacent layer of the conformal coating. Preferably, the covalent linkage between adjacent layers, or between the surface of the cell and the layer adjacent to the surface of the cell involves a linkage containing a substituted triazole group. In some forms, all the layers of the conformal coating are formed from particles (e.g. nanoparticles). In some forms, all the layers of the conformal coating are formed from macromolecules. Preferably, the macromolecules and particles (e.g. nanoparticles) contain zwitterionic polymers (such as zwitterionic polymers containing a sulfobetaine moiety, a carboxybetaine moiety, a phosphoryl choline moiety, or a combination thereof).

Further details on each of the components of the surface-modified cell are provided below.

### (1) Cells

The cell type chosen for surface modification depends on the desired therapeutic effect. The cells may be from the patient (autologous cells), from another donor of the same species (allogeneic cells), or from another species (xenogeneic). Cells can be obtained from biopsy or excision of the patient or a donor, cell culture, or cadavers.

Cells whose surfaces are to be modified can be generally secretory or metabolic cells, i.e., they secrete a therapeutic factor or metabolize toxins, or both; immune cells, e.g., B cells, T cells; metabolic cells, i.e., they metabolize toxic substances; or structural cells, e.g., skin, muscle, blood vessel. In some forms, the cells are naturally secretory, such as islet cells that naturally secrete insulin, adipocytes that secrete hormones such as adiponectin and leptin; or naturally metabolic, such as hepatocytes that naturally detoxify and secrete. In some forms, the cells are bioengineered to express a recombinant protein, such as a secreted protein or metabolic enzyme. Depending on the cell type, the cells may be organized as single cells; cell aggregates or clusters; spheroids; or even natural or bioengineered tissue. Preferably, the cells are secretory cells, immune cells, or metabolic cells.

In some forms, the cells secrete a therapeutically effective substance, such as a protein or nucleic acid. In some forms, the cells metabolize toxic substances. In some forms, the cells are natural, such as islet cells that naturally secrete insulin, or hepatocytes that naturally detoxify. In some forms, the cells are B cells and/or T cells. In some forms, the cells are genetically engineered to express a heterologous protein or nucleic acid and/or overexpress an endogenous protein or nucleic acid. In some forms, the cells form structural tissues, such as skin, bone, cartilage, blood vessels, or muscle.

Preferably, after administration, the cell can be viable for periods such as one day, two days, three days, five days, six days, one week, two weeks, one month, two months, three months, six months, etc.

### (2) Conformal Coating

The conformal coating contains one or more layers, preferably, two or more layers containing particles (e.g. nanoparticles), macromolecules, or both. In some forms, the number of layers in the conformal coating controls the residence time of the surface-modified cell, as determined by confocal fluorescence imaging that provides a readout of coating coverage on the surface of a cell. Preferably, the conformal coating is biocompatible, such that it reduces and/or eliminates eliciting a host immune response against the surface-modified cell or does not result in substantial cytotoxicity as determined using live/dead cell assays (e.g. a cell viability assay using 3-(4,5-Dimethylthiazol-2-yl)-2,5-diphenyltetrazolium bromide). Substantial cytotoxicity can occur when more than 50%, 60%, 70%, 80%, 90%, or 90% of the cell population die in the cell viability assay. Preferably, the conformal coating is stable over a range of pH values, such that the surface-modified cell can perform its desired function at different locations within a host, and is not limited by the specific pH of the location. The materials included in the macromolecules and/or particles can be selected to confer the desired pH stability. Preferably, the duration of the stability of the conformal coating is at least that of the viability of the cell. Accordingly, the stability of the conformal coating is at least one day, two days, three days, five days, six days, one week, two weeks, one month, two months, three months, six months, etc.

At least about 100%, 99%, 98%, 97%, 96%, 95%, 90%, 80%, 70%, 60%, or 50% of the cell's surface can be coated. Preferably, the conformal coating is permeable to a waste product of the cell; oxygen; nutrients; and/or therapeutic substances secreted by the cell, such as insulin; but remains impermeable to a host's antibodies and immune cells. Preferably, the conformal has a thickness that allows diffusion of oxygen, nutrients, and/or the cell's waste products to maintain viability of the cell for protracted time periods. In some forms, the thicknesses of the conformal coating can range between about 0.1 nm and about 5 μm, 1 nm and about 5 μm, between about 0.1 nm and about 1000 nm, between about 10 nm and about 1000 nm, between about 50 nm and about 1000 nm, and between about 50 nm and 600 nm. The thickness of the coating can be governed by the specific cell type, configuration of cells (e.g. single cells versus cluster of cells), and application. For example, a thick conformal coating can be preferred for cell clusters, and a thin conformal coating can be preferred for individual cells. For cells and applications where tropism is desired (e.g. immune cells such as natural killer cells, T cells, macrophages, neutrophils, etc.) or mesenchymal stems cells, a thin conformal coating can be preferred. Examples of a thick conformal coating include conformal coatings with thicknesses of about 100 μm. Examples of a thin conformal coating include conformal coatings with thicknesses of about 10 nm. In some forms, the conformal coating is a degradable coating (triggered by changes in physiological conditions, including pH change, temperature change, and/or localized chemical changes) and system where biological enzymes, such as matrix metalloproteinases, can facilitate degradation of the conformal coating giving rise to cell surface exposure and/or activation of the cell(s).

In some forms, all the layers of the conformal coating contain particles, preferably nanoparticles. In some forms, all the layers of the conformal coating are formed from particles, preferably nanoparticles. In some forms, all the layers of the conformal coating contain macromolecules. In some forms, all the layers of the conformal coating are formed from macromolecules.

### (a) Macromolecules

The macromolecules include a backbone formed via the polymerization of two or more monomers, and optionally containing one or more pendant groups. Preferably, a pendant group is present in at least one of the monomers. Preferably, at least one pendant group contains an abiotic functional group.

### (i) Macromolecule backbone

Preferably, the macromolecules contain a polymer containing a backbone from a poly(acrylate), poly(methacrylate), poly(acrylamide), poly(methacrylamide), poly(vinyl alcohol), poly(ethylene vinyl acetate), poly(vinyl acetate), poly(olefin), poly(ester), poly(hydroxyalkanoates), poly(anhydride), poly (orthoester), polyamide, polyamine, polyether, polyazine, poly(carbonate), polyetheretherketone (PEEK), poly(amino acids), polyimide, polyketal, poly(ketone), polyphosphazine, alginates, polysaccharide, polysiloxane, polysulfone, polyurea, poly(urethane), poly(alkylene oxide), blends, or copolymers thereof. The polymers may be homopolymers, copolymers, or dendrimers (such as PAMAM). The polymer is chosen based on a desired functional group which can be used to couple molecules or chemical moieties, such as the abiotic functional groups herein, to the polymer backbone or to a monomer(s) used to synthesize the functionalized polymer. The polymer can also be chosen based on the desired pH stability of the conformal coating.

### (ii) Abiotic Functional Group

An abiotic functional group can provide for chemical reactions with cell surface molecules, with another functional group (e.g. an abiotic functional group) on the surface of a particle, or with other macromolecules containing another functional group (e.g. an abiotic functional group), while avoiding or minimizing reactions with functional groups that are naturally present on the extracellular membrane of a cell. The abiotic functional group to be conjugated to the macromolecules is selected, such that it more readily initiates and completes a chemical reaction with another abiotic functional group in a cell surface molecule, compared to a functional group that is naturally present on the surface of a native plasma membrane.

The pendant group containing the abiotic functional group can be represented by the formula:

d-R₁—Y, Formula I

d is the point of covalent linkage of the pendant group to the backbone of the polymer, and Y is an abiotic functional group.

In some forms, R₁ is alkyl, substituted alkyl, alkenyl, substituted alkenyl, alkynyl, substituted alkynyl, aryl, substituted aryl, heteroaryl, substituted heteroaryl, alkoxy, substituted alkoxy, aroxy, substituted aroxy, alkylthio, substituted alkylthio, arylthio, substituted arylthio, carbonyl, substituted carbonyl, carboxyl, substituted carboxyl, amino, substituted amino, amido, substituted amido, sulfonyl, substituted sulfonyl, sulfamoyl, substituted sulfamoyl, phosphonyl, substituted phosphonyl, polyaryl, substituted polyaryl, C₃-C₂₀ cyclic, substituted C₃-C₂₀ cyclic, heterocyclic, substituted heterocyclic, amino acid, poly(ethylene glycol), poly(lactic-co-glycolic acid), peptide, or polypeptide group.

In some forms, R₁ is -Aq-unsubstituted C₁-C₁₀ alkylene-Bq-unsubstituted C₁-C₁₀ alkylene-, Aq-unsubstituted C₁-C₁₀ alkylene-Bq-substituted C₁-C₁₀ alkylene-, -Aq-substituted C₁-C₁₀ alkylene-Bq q-unsubstituted C₁-C₁₀ alkylene-, or Aq-substituted C₁-C₁₀ alkylene-Bq-substituted C₁-C₁₀ alkylene-, wherein Aq and Bq are independently —C(O)O—, —C(O)NH—, —OC(O)—, —NHC(O)—, —O—, —NH—NHC(O)—, —OC(O)NH—, —NHC(O)O—, —C(O)—, —OC(O)O—, —S(═O₂)₂—, —S(═O)—, —S—, —N═N—, or —N═CH—.

In some forms, R₁ is -Aq-unsubstituted C₁-C₅ alkylene-Bq-unsubstituted C₁-C₅ alkylene-, Aq-unsubstituted C₁-C₅ alkylene-Bq-substituted C₁-C₅ alkylene-, -Aq-substituted C₁-C₅ alkylene-Bq-unsubstituted C₁-C₅ alkylene-, or Aq-substituted C₁-C₅ alkylene-Bq-substituted C₁-C₅ alkylene-, wherein Aq and Bq are independently —C(O)O—, —C(O)NH—, —OC(O)—, —NHC(O)—, —O—, —NH—NHC(O)—, —OC(O)NH—, —NHC(O)O—, —C(O)—, —OC(O)O—, —S(═O₂)₂—, —S(═O)—, —S—, —N═N—, or —N═CH—.

In some forms, R₁ is —C(O)O-unsubstituted C₂ alkylene-NHC(O)-unsubstituted C₄ alkylene-, —C(O)O-unsubstituted C₂ alkylene-NHC(O)-substituted C₄ alkylene-, —C(O)O-substituted C₂ alkylene-NHC(O)-unsubstituted C₄ alkylene-, or —C(O)O-substituted C₂ alkylene-NHC(O)-substituted C₄ alkylene-.

In some forms, Y is selected from azide; alkyne; alkene; triarylphosphine; aminooxy; carbonyl; hydrazide; sulfonyl chloride; maleimide; aziridine; —CN; acryloyl; acrylamide; sulfone; vinyl sulfone; cyanate; thiocyanate; isocyanate; isothiocyanate; alkoxysilane; dialkyl dialkoxysilane; diaryl dialkoxysilane; trialkyl monoalkoxysilane; vinyl silane; acetohydrazide; acyl azide; acyl halides; epoxide; glycidyl; carbodiimides; thiol; amine; phosphoramidate; vinyl ether; substituted hydrazine; an alkylene glycol bis(diester), e.g. ethylene glycol bis(succinate); thioester, e.g., alkyl thioester, α-thiophenylester, allyl thioester (e.g., allyl thioacetae, allyl thioproprionate); allyl ester (e.g., allyl acetate, allyl propionate); aryl acetate (e.g. phenacyl ester); orthoester; sulfonamide, e.g. 2-N-acyl nitrobenzenesulfonamide; vinyl sulfide; or a combination thereof.

Preferably, the abiotic functional group (Y) is selected from an azide and an alkyne.

### (iii) Weight Average Molecular Weight

The weight average molecular weight of the macromolecules can vary. In some forms, the weight average molecular weight of the macromolecule, as determined by size exclusion chromatography (SEC), can be between about 500 Daltons and about 500,000 Daltons between about 2,000 Daltons and about 300,000 Daltons, between about 5,000 Daltons and about 200,000 Daltons, between about 500 Daltons and about 50,000 Daltons, between about 2,000 Daltons and about 30,000 Daltons, between about 5,000 Daltons and about 20,000 Daltons. The weight average molecular weights of the macromolecules can also depend on their degree of polymerization. In some forms, degree of polymerization is between about 2 and about 10,000, inclusive, between about 2 and about 5,000, inclusive, between about 5 and about 1,000, inclusive, between about 5 and about 500, inclusive, between about 10 and about 200, inclusive, or between about 20 and about 80, inclusive.

### (iv) Zwitterionic Polymers

Preferably, the polymers contain zwitterionic polymers or copolymers thereof, functionalized with an abiotic functional group, as described above. In some forms, the synthetic polymers are zwitterionic polymers. Zwitterionic monomers in the polymers contain a zwitterionic moiety such as a carboxybetaine moiety, a sulfobetaine moiety, a phosphoryl choline moiety, or a combination thereof.

The zwitterionic moiety can be represented by:

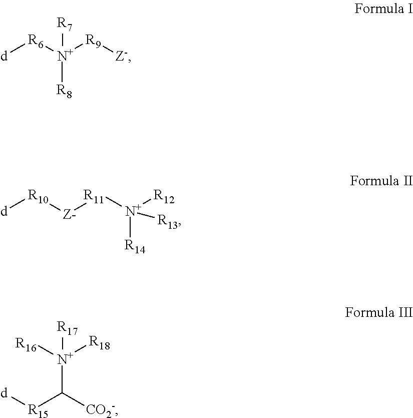

wherein d is the point of covalent linkage of the zwitterion to the backbone of the polymer. In some forms, Z can be a carboxylate, phosphate, phosphonic, phosphanate, sulfate, sulfinic, or sulfonate.

In some forms, R₆-R₁₈ are unsubstituted C₁-C₅ alkyl, substituted C₁-C₅ alkyl, unsubstituted C₁-C₅ alkenyl, substituted C₁-C₅ alkylene, unsubstituted C₁-C₅ alkylene, substituted C₁-C₅ alkenyl, unsubstituted C₁-C₅ alkynyl, substituted C₁-C₅ alkynyl, unsubstituted aryl, substituted aryl, unsubstituted heteroaryl, substituted heteroaryl, unsubstituted C₁-C₅ alkoxy, substituted C₁-C₅ alkoxy, unsubstituted aroxy, substituted aroxy, unsubstituted C₁-C₅ alkylthio, substituted C₁-C₅ alkylthio, unsubstituted arylthio, substituted arylthio, unsubstituted C₁-C₅ carbonyl, substituted C₁-C₅ carbonyl, unsubstituted C₁-C₅ carboxyl, substituted C₁-C₅ carboxyl, unsubstituted C₁-C₅ amino, substituted C₁-C₅ amino, unsubstituted C₁-C₅ amido, substituted C₁-C₅ amido, unsubstituted C₁-C₅ sulfonyl, substituted C₁-C₅ sulfonyl, unsubstituted C₁-C₅ sulfamoyl, substituted C₁-C₅ sulfamoyl, unsubstituted C₁-C₅ phosphonyl, substituted C₁-C₅ phosphonyl, unsubstituted polyaryl, substituted polyaryl, unsubstituted C₃-C₁₀ cyclic, substituted C₃-C₁₀ cyclic, unsubstituted C₃-C₁₀ heterocyclic, or substituted C₃-C₂₀ heterocyclic.

In some forms, R₆, R₉, R₁₀, R₁₁ and R₁₅, are independently unsubstituted C₁-C₅ alkyl, substituted C₁-C₅ alkyl, substituted C₁-C₅ alkylene, or unsubstituted C₁-C₅ alkylene, C₁-C₅ alkoxy, substituted C₁-C₅ alkoxy, unsubstituted aroxy, substituted aroxy, unsubstituted C₁-C₅ alkylthio, substituted C₁-C₅ alkylthio, unsubstituted arylthio, substituted arylthio, unsubstituted C₁-C₅ carbonyl, substituted C₁-C₅ carbonyl, unsubstituted C₁-C₅ carboxyl, substituted C₁-C₅ carboxyl, unsubstituted C₁-C₅ amino, substituted C₁-C₅ amino, unsubstituted C₁-C₅ amido, substituted C₁-C₅ amido, unsubstituted C₁-C₅ sulfonyl, substituted C₁-C₅ sulfonyl, unsubstituted C₁-C₅ sulfamoyl, substituted C₁-C₅ sulfamoyl, unsubstituted C₁-C₅ phosphonyl, or substituted C₁-C₅ phosphonyl.

In some forms, R₇, R₈, R₁₂, R₁₃, R₁₄, R₁₆, R₁₇, and R₁₈, are independently hydrogen, unsubstituted C₁-C₅ alkyl, or substituted C₁-C₅ alkyl.

In some forms, the zwitterionic moieties can be:

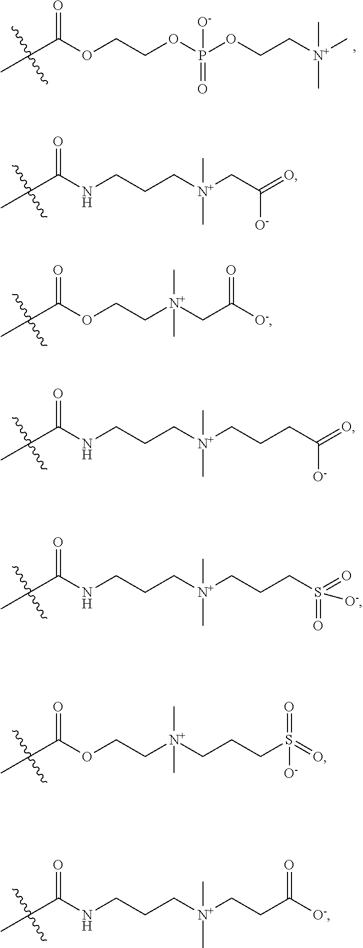

or combinations thereof.

In some forms, the zwitterionic polymers can be a structure shown below:

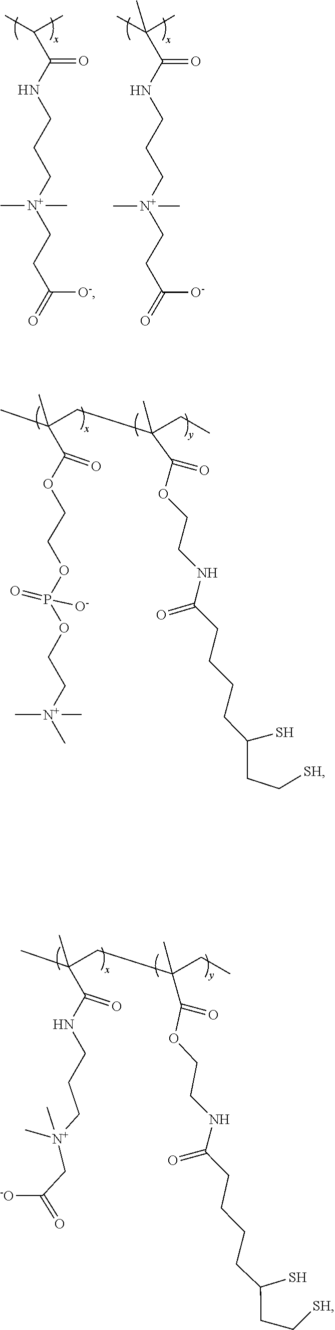

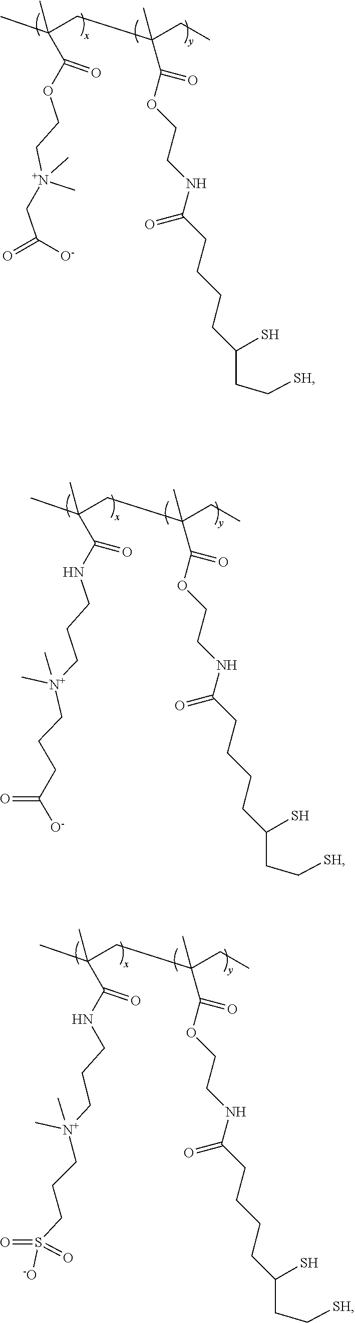

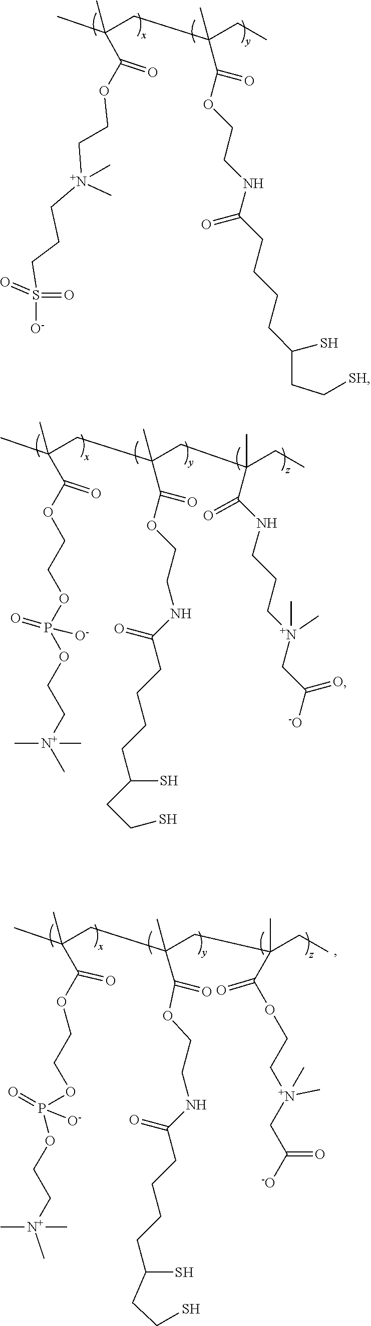

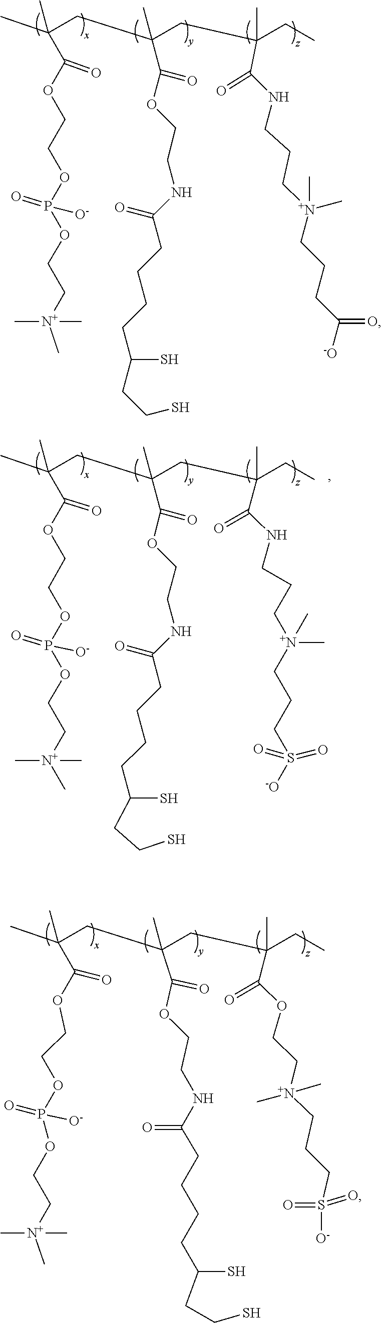

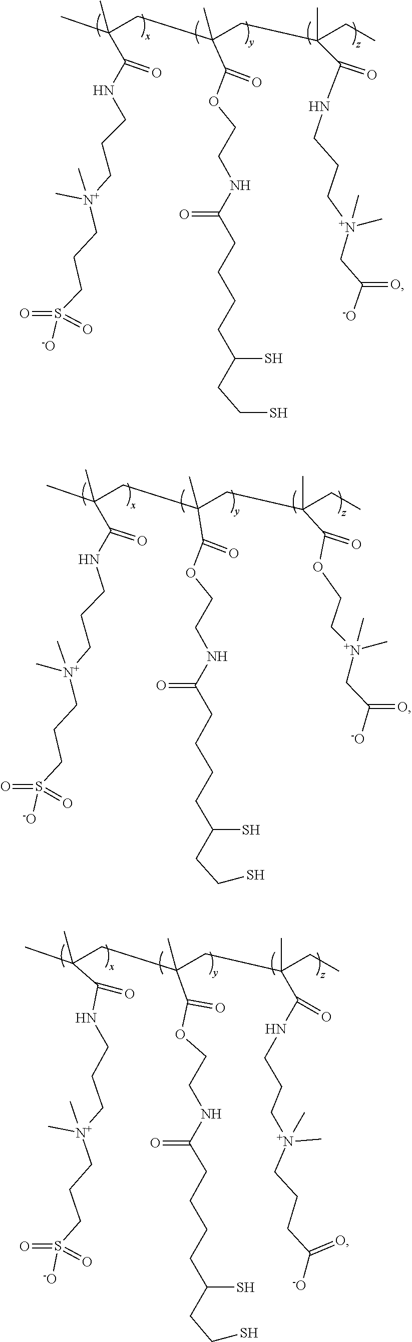

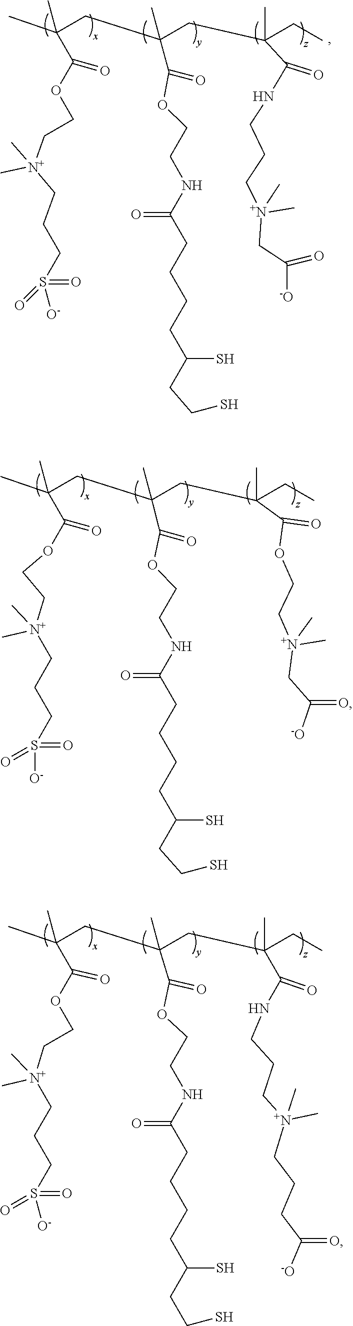

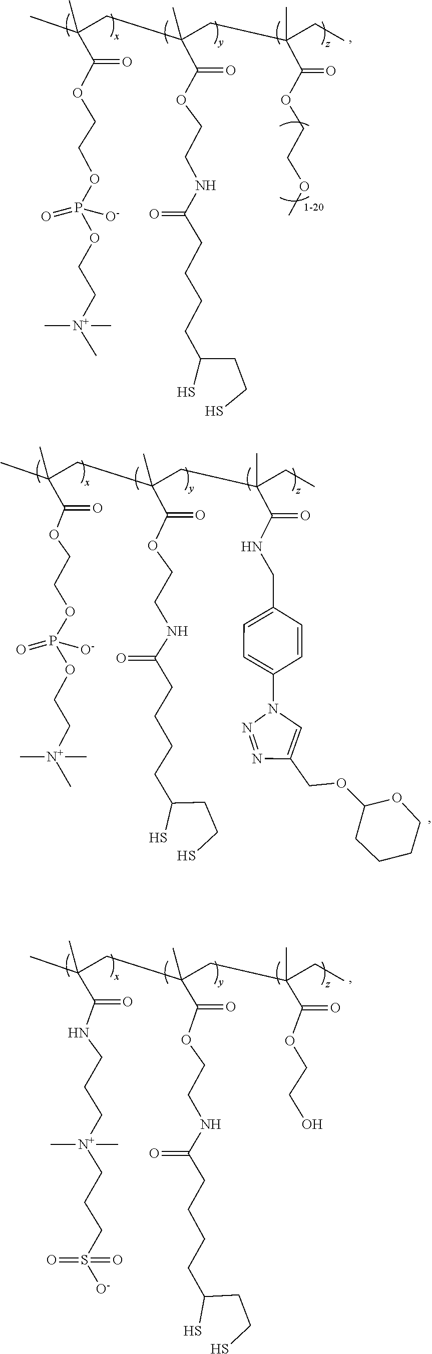

or a combination thereof. x is independently an integer between 1 and 1,000, inclusive, preferably between 10 and 200, inclusive. y is an integer between 1 and 1,000, inclusive, preferably between 10 and 200, inclusive. z is independently an integer between 1 and 1,000, inclusive, preferably between 10 and 200, inclusive. z can be zero.

### (v) Modified Alginates

In some forms, the macromolecule contains a chemically modified alginate.

Preferably in these forms, the backbone of the macromolecule contains an alginate that has been modified, such that one or more of the monomers of the alginate contain a pendant group having Formula I:

d-R₁—Y, Formula I

wherein d, R₁, and Y are as described above for Formula I.

Preferably, the monomer is a covalently modified monomer defined by Formula IV

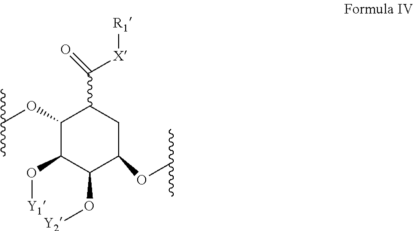

wherein,

X′ is oxygen, sulfur, or NR₄′;

R₁′ is, independently in the one or more modified monomers, hydrogen, alkyl, substituted alkyl, alkenyl, substituted alkenyl, alkynyl, substituted alkynyl, phenyl, substituted phenyl, aryl, substituted aryl, heteroaryl, substituted heteroaryl, alkoxy, substituted alkoxy, phenoxy, substituted phenoxy, aroxy, substituted aroxy, alkylthio, substituted alkylthio, phenylthio, substituted phenylthio, arylthio, substituted arylthio, carbonyl, substituted carbonyl, carboxyl, substituted carboxyl, amino, substituted amino, amido, substituted amido, sulfonyl, substituted sulfonyl, sulfonic acid, phosphoryl, substituted phosphoryl, phosphonyl, substituted phosphonyl, polyaryl, substituted polyaryl, C₃-C₂₀ cyclic, substituted C₃-C₂₀ cyclic, heterocyclic, substituted heterocyclic, amino acid, poly(ethylene glycol), peptide, polypeptide group; or Formula I, as defined above;

Y₁′ and Y₂′ are independently hydrogen, —PO(OR₅′)₂, or Formula I; or

Y₂′ is absent, and Y₁′, together with the two oxygen atoms to which Y₁′ and Y₂′ are attached form a cyclic structure as shown in Formula V

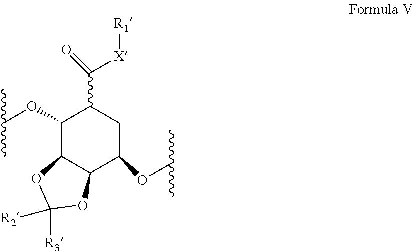

R₂′ and R₃′ are, independently, hydrogen, alkyl, substituted alkyl, alkenyl, substituted alkenyl, alkynyl, substituted alkynyl, phenyl, substituted phenyl, aryl, substituted aryl, heteroaryl, substituted heteroaryl, alkoxy, substituted alkoxy, phenoxy, substituted phenoxy, aroxy, substituted aroxy, alkylthio, substituted alkylthio, phenylthio, substituted phenylthio, arylthio, substituted arylthio, carbonyl, substituted carbonyl, carboxyl, substituted carboxyl, amino, substituted amino, amido, substituted amido, polyaryl, substituted polyaryl, C₃-C₂₀ cyclic, substituted C₃-C₂₀ cyclic, heterocyclic, substituted heterocyclic, amino acid, poly(ethylene glycol), peptide, or polypeptide group; or R₂′ and R₃′, together with the carbon atom to which they are attached, form a 3- to 8-membered unsubstituted or substituted carbocyclic or heterocyclic ring; and

R₄′ and R₅′ are, independently, hydrogen, alkyl, substituted alkyl, alkenyl, substituted alkenyl, alkynyl, substituted alkynyl, phenyl, substituted phenyl, aryl, substituted aryl, heteroaryl, substituted heteroaryl, alkoxy, substituted alkoxy, phenoxy, substituted phenoxy, aroxy, substituted aroxy, alkylthio, substituted alkylthio, phenylthio, substituted phenylthio, arylthio, substituted arylthio, carbonyl, substituted carbonyl, carboxyl, substituted carboxyl, amino, substituted amino, amido, substituted amido, polyaryl, substituted polyaryl, C₃-C₂₀ cyclic, substituted C₃-C₂₀ cyclic, heterocyclic, substituted heterocyclic, amino acid, poly(ethylene glycol), peptide, or polypeptide group;

with the proviso that at least one of R₁′ Y₁′, R₂′ and R₃′ can be Formula I.

Preferably, the modified alginate can also contain one or more covalently modified monomers defined by Formula VI

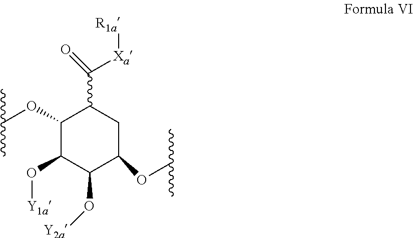

wherein,

Xₐ′ is oxygen, sulfur, or NR₄ₐ′;

R₁ₐ′ is, independently in the one or more modified monomers hydrogen, alkyl, substituted alkyl, alkenyl, substituted alkenyl, alkynyl, substituted alkynyl, phenyl, substituted phenyl, aryl, substituted aryl, heteroaryl, substituted heteroaryl, alkoxy, substituted alkoxy, phenoxy, substituted phenoxy, aroxy, substituted aroxy, alkylthio, substituted alkylthio, phenylthio, substituted phenylthio, arylthio, substituted arylthio, carbonyl, substituted carbonyl, carboxyl, substituted carboxyl, amino, substituted amino, amido, substituted amido, sulfonyl, substituted sulfonyl, sulfonic acid, phosphoryl, substituted phosphoryl, phosphonyl, substituted phosphonyl, polyaryl, substituted polyaryl, C₃-C₂₀ cyclic, substituted C₃-C₂₀ cyclic, heterocyclic, substituted heterocyclic, amino acid, poly(ethylene glycol), peptide, or polypeptide group;

Y₁ₐ′ and Y₂ₐ′ are independently hydrogen, —PO(OR₅ₐ′)₂; or

Y₂ₐ′ is absent, and Y₁ₐ′, together with the two oxygen atoms to which Y₁ₐ′ and Y₂ₐ′ are attached form a cyclic structure as shown in Formula VII

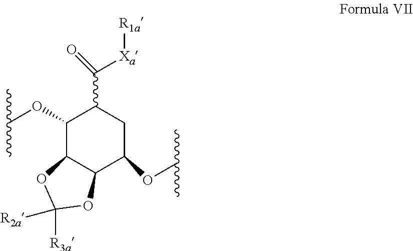

R₂ₐ′ and R₃ₐ′ are, independently, hydrogen, alkyl, substituted alkyl, alkenyl, substituted alkenyl, alkynyl, substituted alkynyl, phenyl, substituted phenyl, aryl, substituted aryl, heteroaryl, substituted heteroaryl, alkoxy, substituted alkoxy, phenoxy, substituted phenoxy, aroxy, substituted aroxy, alkylthio, substituted alkylthio, phenylthio, substituted phenylthio, arylthio, substituted arylthio, carbonyl, substituted carbonyl, carboxyl, substituted carboxyl, amino, substituted amino, amido, substituted amido, polyaryl, substituted polyaryl, C₃-C₂₀ cyclic, substituted C₃-C₂₀ cyclic, heterocyclic, substituted heterocyclic, amino acid, poly(ethylene glycol), peptide, or polypeptide group; or R₂ₐ′ and R₃ₐ′, together with the carbon atom to which they are attached, form a 3- to 8-membered unsubstituted or substituted carbocyclic or heterocyclic ring; and

R₄ₐ′ and R₅ₐ′ are, independently, hydrogen, alkyl, substituted alkyl, alkenyl, substituted alkenyl, alkynyl, substituted alkynyl, phenyl, substituted phenyl, aryl, substituted aryl, heteroaryl, substituted heteroaryl, alkoxy, substituted alkoxy, phenoxy, substituted phenoxy, aroxy, substituted aroxy, alkylthio, substituted alkylthio, phenylthio, substituted phenylthio, arylthio, substituted arylthio, carbonyl, substituted carbonyl, carboxyl, substituted carboxyl, amino, substituted amino, amido, substituted amido, polyaryl, substituted polyaryl, C₃-C₂₀ cyclic, substituted C₃-C₂₀ cyclic, heterocyclic, substituted heterocyclic, amino acid, poly(ethylene glycol), peptide, or polypeptide group.

In some forms of Formula VI, R₁ₐ′ has the structure:

-Az-Bz-(-Cz)δ, Formula XIII

wherein δ is an integer between 0 and 10, inclusive, preferably δ is 1.

In some forms of Formula XIII, Az can be

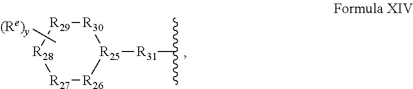

wherein in Formula XIV of Az: R₃₁—(CR₃₂R₃₂)ₚ—; p is an integer from 0 to 5; each R₃₂ is hydrogen, unsubstituted alkyl, or substituted alkyl; each R^{e} is independently unsubstituted alkyl, substituted alkyl, unsubstituted alkenyl, unsubstituted alkenyl, unsubstituted alkynyl, substituted alkynyl, unsubstituted alkoxy, substituted alkoxy, unsubstituted alkylamino, substituted alkylamino, unsubstituted dialkylamino, substituted dialkylamino, hydroxy, unsubstituted aryl, substituted aryl, unsubstituted heteroaryl, substituted heteroaryl, unsubstituted carboxyl, substituted carboxyl, unsubstituted amino, substituted amino, unsubstituted amido, substituted amido, unsubstituted C₃-C₂₀ cyclic, substituted C₃-C₂₀ cyclic, unsubstituted C₃-C₂₀ heterocyclic, or substituted C₃-C₂₀ heterocyclic; y is an integer between 0 and 11, inclusive; R₂₅, R₂₆, R₂₇, R₂₈, R₂₉, and R₃₀ are independently C or N, wherein the bonds between adjacent R₂₅ to R₃₀ are double or single according to valency, and wherein R₂₅ to R₃₀ are bound to none, one, or two hydrogens according to valency, or

Az can be

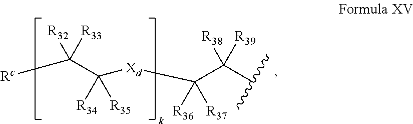

wherein in Formula XV of Az, R₃₂, R₃₃, R₃₄, R₃₅, R₃₆, R₃₇, R₃₈, and R₃₉ are independently hydrogen, unsubstituted alkyl, substituted alkyl, unsubstituted alkenyl, substituted alkenyl, unsubstituted alkynyl, substituted alkynyl, unsubstituted phenyl, substituted phenyl, unsubstituted aryl, substituted aryl, unsubstituted heteroaryl, substituted heteroaryl, unsubstituted arylalkyl, substituted arylalkyl, unsubstituted alkoxy, substituted alkoxy, unsubstituted aroxy, substituted aroxy, unsubstituted carbonyl, substituted carbonyl, unsubstituted carboxyl, substituted carboxyl, unsubstituted amino, substituted amino, unsubstituted amido, substituted amido, unsubstituted C₃-C₂₀ cyclic, substituted C₃-C₂₀ cyclic, unsubstituted C₃-C₂₀ heterocyclic, substituted C₃-C₂₀ heterocyclic, poly(ethylene glycol), or poly(lactic-co-glycolic acid); k is an integer from 0 to 20; each X_{d} is independently absent, O, or S; and RC is Bz.

In some forms of Formula XIII and Formula XV, wherein Bz can be

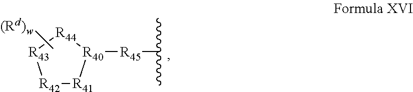

wherein for Formula XVI of Bz, R₄₅ is —(CR₄₆R₄₆)ₚ—; p is an integer from 0 to 5; each R₄₆ is hydrogen, unsubstituted alkyl, or substituted alkyl; each R^{d} is independently unsubstituted alkyl, substituted alkyl, unsubstituted alkenyl, unsubstituted alkenyl, unsubstituted alkynyl, substituted alkynyl, unsubstituted alkoxy, substituted alkoxy, unsubstituted alkylamino, substituted alkylamino, unsubstituted dialkylamino, substituted dialkylamino, hydroxy, unsubstituted aryl, substituted aryl, unsubstituted heteroaryl, substituted heteroaryl, unsubstituted carboxyl, substituted carboxyl, unsubstituted amino, substituted amino, unsubstituted amido, substituted amido, unsubstituted C₃-C₂₀ cyclic, substituted C₃-C₂₀ cyclic, unsubstituted C₃-C₂₀ heterocyclic, or substituted C₃-C₂₀ heterocyclic; w is an integer between 0 and 4, inclusive; each R₄₀, R₄₁, R₄₂, R₄₃, and R₄₄, are independently C or N, wherein the bonds between adjacent R₄₀ to R₄₄ are double or single according to valency, and wherein R₄₀ to R₄₄ are bound to none, one, or two hydrogens according to valency.

In some forms of Formula XIII, Cz can be

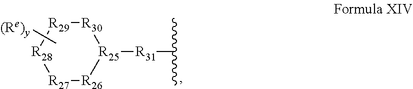

wherein in Formula XIV of Cz, R₃₁ is —(CR₃₂R₃₂)ₚ— or —(CR₃₂R₃₂)ₚ—X_{b}—(CR₃₂R₃₂)_{q}—; p and q are independently integers between 0 to 5, inclusive; each R₃₂ is hydrogen, unsubstituted alkyl, or substituted alkyl; X_{b} is absent, —O—, —S—, —S(O)—, —S(O)₂—, or NR_{47;} R₄₇ is unsubstituted alkyl or substituted alkyl; each Re is independently unsubstituted alkyl, substituted alkyl, unsubstituted alkenyl, unsubstituted alkenyl, unsubstituted alkynyl, substituted alkynyl, unsubstituted alkoxy, substituted alkoxy, unsubstituted alkylamino, substituted alkylamino, unsubstituted dialkylamino, substituted dialkylamino, hydroxy, unsubstituted aryl, substituted aryl, unsubstituted heteroaryl, substituted heteroaryl, unsubstituted carboxyl, substituted carboxyl, unsubstituted amino, substituted amino, unsubstituted amido, substituted amido, unsubstituted C₃-C₂₀ cyclic, substituted C₃-C₂₀ cyclic, unsubstituted C₃-C₂₀ heterocyclic, or substituted C₃-C₂₀ heterocyclic; y is an integer between 0 and 11, inclusive; R₂₅, R₂₆, R₂₇, R₂₈, R₂₉, and R₃₀ are independently C or N, wherein the bonds between adjacent R₂₅ to R₃₀ are double or single according to valency, and wherein R₂₅ to R₃₀ are bound to none, one, or two hydrogens according to valency.

In some forms, for Formula XIV of Az, each R₃₂ is hydrogen, and p is 1.

In some forms, for Formula XIV of Az, each R₃₂ is hydrogen, p is 1, R₂₅ is C, and R₂₆—R₃₀ are CH, and the bonds between R₂₅ and R₂₆, between R₂₇ and R₂₈, and between R₂₀ and R₃₀ are double bonds.

In some forms, for Formula XIV of Az, each R₃₂ is hydrogen, p is 1, R₂₅ is C, and R₂₆—R₃₀ are CH, and the bonds between R₂₅ and R₂₆, between R₂₇ and R₂₈, and between R₂₀ and R₃₀ are double bonds, and y is 1.

In some forms, for Formula XIV of Az, each R₃₂ is hydrogen, p is 1, R₂₅ is C, and R₂₆—R₃₀ are CH, and the bonds between R₂₅ and R₂₆, between R₂₇ and R₂₈, and between R₂₀ and R₃₀ are double bonds, y is 1, and R^{c} is Bz having Formula XVI.

In some forms, for Formula XIV of Az, each R₃₂ is hydrogen, p is 1, R₂₅ is C, and R₂₆—R₃₀ are CH, and the bonds between R₂₅ and R₂₆, between R₂₇ and R₂₈, and between R₂₀ and R₃₀ are double bonds, y is 1, and for Formula XVI of Bz, p is 0.

In some forms, for Formula XIV of Az, each R₃₂ is hydrogen, p is 1, R₂₅ is C, and R₂₆—R₃₀ are CH, and the bonds between R₂₅ and R₂₆, between R₂₇ and R₂₈, and between R₂₀ and R₃₀ are double bonds, y is 1, and for Formula XVI of Bz, p is 0, and R₄₀-R₄₂ are N.

In some forms, for Formula XIV of Az, each R₃₂ is hydrogen, p is 1, R₂₅ is C, and R₂₆—R₃₀ are CH, and the bonds between R₂₅ and R₂₆, between R₂₇ and R₂₈, and between R₂₉ and R₃₀ are double bonds, y is 1, and for Formula XVI of Bz, p is 0, R₄₀—R₄₂ are N, and R₄₃ and R₄₄ are C.

In some forms, for Formula XIV of Az, each R₃₂ is hydrogen, p is 1, R₂₅ is C, and R₂₆—R₃₀ are CH, and the bonds between R₂₅ and R₂₆, between R₂₇ and R₂₈, and between R₂₀ and R₃₀ are double bonds, y is 1, and Formula XVI of Bz, is

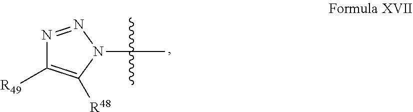

wherein R₄₈ and R₄₉ are independently hydrogen,

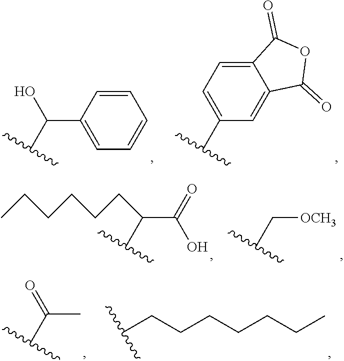

or Cz having Formula XIV.

In some forms, for Formula XIV of Cz, R₃₁ is —(CR₃₂R₃₂)ₚ—X_{b}—(CR₃₂R₃₂)_{q}—, each R₃₂ is hydrogen, and p is 0.

In some forms, for Formula XIV of Cz, R₃₁ is —(CR₃₂R₃₂)ₚ—X_{b}—(CR₃₂R₃₂)_{q}—, each R₃₂ is hydrogen, p is 0, and q is 1.

In some forms, for Formula XIV of Cz, R₃₁ is —(CR₃₂R₃₂)ₚ—X_{b}—(CR₃₂R₃₂)_{q}—, each R₃₂ is hydrogen, p is 0, q is 1, and X_{b} s 0 or —S(O)2—.

In some forms, for Formula XIV of Cz, R₃₁ is —(CR₃₂R₃₂)ₚ—X₆—(CR₃₂R₃₂)_{q}—, each R₃₂ is hydrogen, p is 0, q is 1, X_{b} is 0, and R₂₆ is 0.

In some forms, for Formula XIV of Cz, R₃₁ is —(CR₃₂R₃₂)ₚ—X_{b}—(CR₃₂R₃₂)_{q}—, each R₃₂ is hydrogen, p is 0, q is 1, X_{b} is 0, R₂₆ is 0, and R₂₅ is CH.

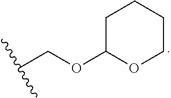 [SMILES: *COC1CCCCO1]

In some forms, Formula XIV of Cz is

In some forms, of Formula XIII, Az can be

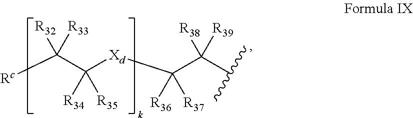

as defined above, and R^{c} is Bz having Formula XVI, as defined above.

In some forms, for Formula IX of Az, Xd is O.

In some forms, for Formula IX of Az, Xd is O, and R₃₂—R₃₉ are hydrogen.

In some forms, for Formula IX of Az, Xd is O, R₃₂—R₃₉ are hydrogen, and k is an integer between 1 and 5, inclusive, preferably 3.

In some forms, for Formula XVI of Bz, p is 0.

In some forms, for Formula XVI of Bz, p is 0, and R₄₀—R₄₂ are N.

In some forms, for Formula XVI of Bz, p is 0, R₄₀—R₄₂ are N, and R₄₃ and R₄₄ are C.

In some forms, Formula XVI of Bz is

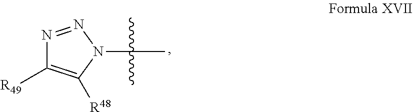

wherein R₄₈ and R₄₉ are independently hydrogen,

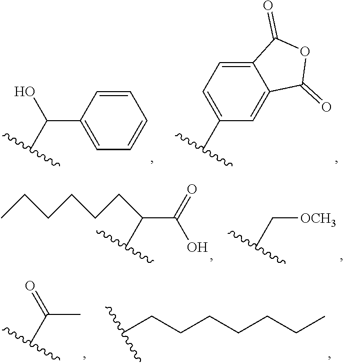

or Cz having Formula XIV.

In some forms, for Formula XIV of Cz, R₃₁ is —(CR₃₂R₃₂)ₚ—, each R₃₂ is hydrogen, p is 0 or 1.

In some forms, for Formula XIV of Cz, R₃₁ is —(CR₃₂R₃₂)ₚ—, each R₃₂ is hydrogen, p is 1, and R₂₅ is N.

In some forms, for Formula XIV of Cz, R₃₁ is —(CR₃₂R₃₂)ₚ—, each R₃₂ is hydrogen, p is 1, R₂₅ is N, and R₂₈ is S(O)₂.

In some forms, for Formula XIV of Cz, R₃₁ is —(CR₃₂R₃₂)ₚ—, each R₃₂ is hydrogen, p is 1, R₂₅ is N, R₂₈ is S(O)_{2,} and R₂₆, R₂₇, R₂₀, and R₃₀ are CH₂.

In some forms, Formula XIV of Cz is

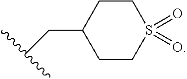 [SMILES: *CC1CCS(=O)(=O)CC1]

In some forms, for Formula XIV of Cz, R₃₁ is —(CR₃₂R₃₂)ₚ—, each R₃₂ is hydrogen, p is 0, R₂₅ is C, R₂₆—R₃₀ are CH, and the bonds between R₂₅ and R₂₆, between R₂₇ and R₂₈, and between R₂₀ and R₃₀ are double bonds.

In some forms, for Formula XIV of Cz, R₃₁ is —(CR₃₂R₃₂)ₚ—, each R₃₂ is hydrogen, p is 0, R₂₅ is C, R₂₆-R₃₀ are CH, the bonds between R₂₅ and R₂₆, between R₂₇ and R₂₈, and between R₂₉ and R₃₀ are double bonds, and y is 1.

In some forms, for Formula XIV of Cz, R₃₁ is —(CR₃₂R₃₂)ₚ—, p is 0, R₂₅ is C, and R₂₆—R₃₀ are CH, the bonds between R₂₅ and R_{26,} between R₂₇ and R₂₈, and between R₂₀ and R₃₀ are double bonds, y is 1, and R^{e} is —NH₂.

In some forms, Formula XIV of Cz is

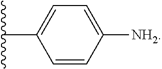 [SMILES: *c1ccc(N)cc1]

In some forms R₁ₐ′ can be

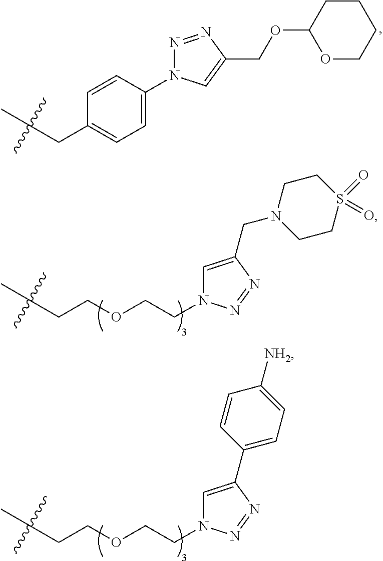

or a combination thereof.

In some forms, Xₐ′—R₁ₐ′ can be

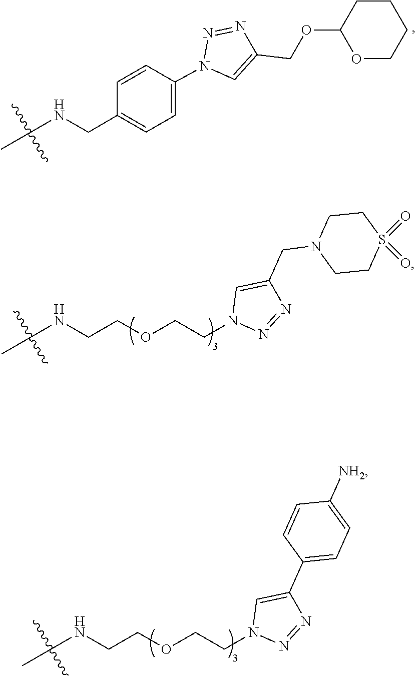

or a combination thereof.

In some forms, the modified alginate can contain a covalently modified monomer, as defined above, of Formula IV, Formula VI, or a combination thereof. Every modified alginate of Formula IV or Formula VI within the above definitions is intended to be and should be considered to be specifically disclosed herein. Further, every subgroup that can be identified within the above definition is intended to be and should be considered to be specifically disclosed herein. As a result, every sub-group, species, or both, that can be identified within the definition of Formula IV and/or Formula VI can be specifically included, excluded, or combined in any combination of sub-group, species, or both of Formula IV and/or Formula VI. As an example, a modified alginate can contain a covalently modified monomer that contains an alkyne, such as a dibenzocyclooctyne, and another covalently modified monomer that contains a triazole-thiomorpholine dioxide moiety, such as:

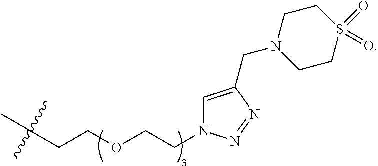

U.S. Patent Application Publication No. 2016/0030360 by Vegas, et al., and U.S. Pat. No. 9,422,373 to Vegas, et al., describe chemically modified alginates. The chemically modified alginates can be further functionalized with the abiotic functional groups, as described above.

### (b) Particles

The particles can include any of the macromolecules described above. In some forms, the particles (e.g. nanoparticles) are formed from the macromolecules described above. Preferably, the particles contain a zwitterionic polymer or a copolymer thereof, or a chemically modified alginate described above.

Generally, the particles have an average diameter between 1 nm and 5 um. When the particles are nanoparticles, the nanoparticles have an average diameter greater than or equal to 1 nm and less than 1 μm, between about 10 nm and about 500 nm, between about 25 nm and about 500 nm, or between about 50 nm and about 200 nm. In some forms, the particles (e.g. nanoparticles) have a zeta potential between about −35 mV and about +35 mV, between about −20 mV and about +20 mV, between about −10 mV and about +10 mV, or between about −5 mV and about +5 mV. Preferably, the abiotic functional groups are displayed on the surface of the particles. Preferably, the particles are nanoparticles. Preferably, the nanoparticles are nanogels. A nanogels can be a nanoparticle formed from polymers that are cross-linked. Preferably, cross-linking results in the formation of a polymer network. The polymers can be hydrophilic polymers or can contain hydrophilic polymers. Preferably, the polymer network is hydrophilic, i.e., a hydrophilic polymer network. The cross-linking can be ionic, covalent, or both.

In a preferred embodiment, the particle contains poly(carboxybetaine acrylamide). Preferably, the poly(carboxybetaine acrylamide) are crosslinked using a linker, such as N,N′-methylene-bisacrylamide.

Preferably, the abiotic functional groups are displayed on the surface of the particles. The abiotic functional groups can be introduced prior to or after particle formation. When introduced prior to particle formation, the abiotic functional group is included in a macromolecule used to form the particle, as discussed above. When introduced post-particle formation, a compound containing the abiotic functional group reacts with a chemical group on the surface of the particle. Examples of compounds that can be used to introduce abiotic functional groups include, but are not limited, to amine-PEG4-DBCO, amine-PEG4-azide, and amine-PEGS-azide, shown below.

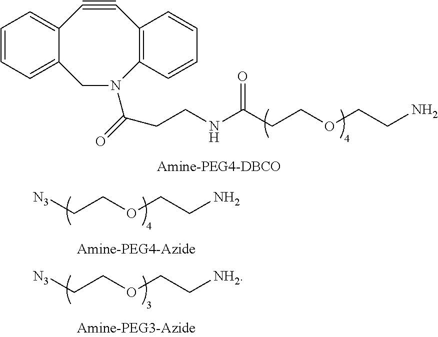

### (3) Linkages

Macromolecules, particles (e.g. nanoparticles), or both, in the layer adjacent to the surface of the cell are conjugated to a cell surface molecule, preferably via a covalent linkage, generally referred to herein as a linker. In some forms, the cell surface molecule is a protein, glycoprotein, lipid, glycolipid, or a combination thereof, preferably a glycoprotein. The cell surface molecules are modified to contain any of the abiotic functional groups described herein. Preferably, an abiotic functional group in a macromolecule is involved in a chemical reaction with an abiotic functional group introduced into a cell surface molecule.

Macromolecules, particles, or both, in a given layer are also conjugated to macromolecules, particles, or both, in an adjacent layer, preferably via covalent linkage, and preferably via abiotic functional groups.

In some forms, the linker is non-cleavable or cleavable. Cleavable linkers include chemically cleavable linkers (e.g. solvolysis (such as hydrolysis), reduction, oxidation, etc.), enzymatically cleavable linkers, thermally cleavable linkers, photocleavable linkers, or a combination thereof. Leriche, et al., Bioorg. Med. Chem. 2012, 20, 571-582, describes cleavable linkers in chemical biology, the contents of which are incorporated herein by reference.

The covalent linkage can include a substituted triazole (azide+alkyne), an amide (azide+triphenylphosphine), a carbamate (amine+hydroxyl using diimidazole carbonyl; or isocynate+hydroxyl), oxime ether (carbonyl+aminooxy), hydrazone (carbonyl+hydrazide), thio-ether (maleimide+thiol), a carbonyl (ketone), imine (carbonyl+amine), sulfonamide (sulfonyl chloride+amine), azo (aromatic diazonium and anilines or phenols), dialkyl dialkoxysilane, diaryl dialkoxysilane, orthoester, acetal, aconityl, β-thiopropionate, phosphoramidate, trityl, vinyl ether, polyketal, or a combination thereof. The entries in parentheses show the functional groups that can be involved in forming the specified covalent linkage.

Preferably, the covalent linkage between the cell surface molecule and the macromolecules, nanoparticles, or both, in the layer adjacent to the surface of the cell includes a substituted triazole. Preferably, the covalent linkage between the macromolecules, nanoparticles, or both, in a given layer, and the macromolecules, nanoparticles, or both, in an adjacent layer includes a substituted triazole.

### (4) Therapeutic, Diagnostic, and Prophylactic Agents

The surface-modified cell can also include therapeutic, diagnostic, and/or prophylactic agents. Preferably, these agents are associated with the conformal coating, i.e., the agents can be distributed within the matrix of the conformal coating; conjugated to macromolecules, nanoparticles, or both, used to form the layers of the conformal coating; or a combination thereof. In the case of nanoparticles, the agents can be encapsulated, on the surface, or both. In some forms, the conformal coating provides for sustained release of the therapeutic, diagnostic, or prophylactic agent.

The inclusion of therapeutic agents in the surface-modified cell, can boost its therapeutic effects. Therapeutic agents include, but are not limited to, small molecules, antibodies, nucleic acids, carbohydrates, chemotherapeutic agents, or combinations thereof. Specific classes of therapeutic agents include, but are not limited to, immunosuppressants, anti-inflammatory agents, anticancer agents, antibiotics, and antiviral agents. A description of these and other classes of useful therapeutic agents and a listing of species within each class can be found in Martindale, The Extra Pharmacopoeia, 30th Ed. (The Pharmaceutical Press, London 1993), the disclosure of which is incorporated herein by reference in its entirety.

A surface-modified cell containing diagnostic agents can result in non-invasive tracking of the surface-modified cell after administration. Exemplary diagnostic agents include, but are not limited to, contrast agents; radionuclides; and fluorescent, luminescent, and magnetic moieties. In some forms, the diagnostic agent is an agent used in magnetic resonance imaging (MRI), such as iron oxide particles or gadolinium complexes. Gadolinium complexes that have been approved for clinical use include gadolinium chelates with DTPA, DTPA-BMA, DOTA and HP-DO3A (reviewed in Aime, et al., Chemical Society Reviews 1998, 27:19). In some forms, the diagnostic agent is an iron oxide nanoparticle. In some forms, the iron oxide nanoparticle is encapsulated in the nanoparticles used to form a layer of conformal coating.

In some forms, a prophylactic agent is included in the surface-modified cell. In these forms, the prophylactic agent includes a vaccine. Vaccines can be isolated proteins, peptides, carbohydrates, or glycoproteins; inactivated organisms and viruses; dead organisms and virus; genetically altered organisms or viruses; and cell extracts.

### (5) Targeting Agents

Modification of cell surfaces with targeting agents will enhance tissue specific homing of the surface-modified cells after administration. Therefore, in some forms, the macromolecules, nanoparticles, or both, include a targeting agent. Preferably, the targeting agent is displayed on the outermost layer of the conformal coating, by covalently or non-covalently attaching the targeting agent to the macromolecules, nanoparticles, or both, used to form the outermost layer. Exemplary targeting agents include, but are not limited to, antibodies and antigen binding fragments thereof, aptamers, peptides, and small molecules. In the case of nanoparticles, preferably, the targeting agent is displayed on the surface of the nanoparticle. Typically, the targeting agents have an affinity for a cell-surface receptor or cell-surface antigen on the target cells or tissue.

Preferably, the target molecule is associated with a disease or preferentially over-expressed in a diseased tissue or cell compared to a non-diseased tissue or cell. The target molecule can be a cell surface protein, glycoprotein, lipid, or glycolipid. In some forms, the target molecule can be a receptor that is selectively expressed on a specific cell surface, a tissue or an organ.

### III. Methods of Making

### (1) Macromolecules

Any suitable method known in the art can be used to generate the macromolecules from their respective monomers. Suitable methods include, but are not limited to, radical polymerization (e.g. reversible addition-fragmentation chain transfer); anionic polymerization; cationic polymerization; isolation and purification of a naturally occurring polymer, such as an alginate; and enzymatic polymerization. In some forms, the monomers used to generate the macromolecule contain a monomer with a pendant group that contains an abiotic functional group prior to polymerization. In some forms, the macromolecule is formed first, followed by functionalization of the macromolecule to introduce the abiotic functional group.

### (2) Nanoparticles

Any of the macromolecules described above, can be used to make the nanoparticles. It can be beneficial to first form the nanoparticles, followed by functionalization to introduce abiotic functional groups on its surface, in order to avoid possible waste of due to pre-functionalized macromolecules having abiotic functional groups buried in the core of the nanoparticles.

The nanoparticles described herein can be formed using a variety of techniques known in the art. The technique to be used can depend on a variety of factors including the polymer used to form the nanoparticles, the desired size range of the resulting nanoparticles, and suitability for the therapeutic, diagnostic, and/or prophylactic agent to be incorporated. Suitable techniques include, but are not limited to:

### (a) Crosslinking

In some forms, the nanoparticles are formed by crosslinking the macromolecules described above via covalent bonds using crosslinkers; ionic crosslinking; or both. In the case of covalent crosslinking, the crosslinkers can be charged, neutral, zwitterionic, bivalent (i.e., contain two reactive moieties), multivalent (i.e., contain three or more reactive moieties), homofunctional (i.e., contain the same reactive moieties), heterofunctional (i.e., contain the different reactive moieties), or a combination thereof. Ionic crosslinking can be performed using multivalent cations, polyelectrolytes (i.e., dimers, oligomers, or polymers with a net charge greater than one). Crosslinking can give rise to the formation of gel capsules, such as nanogels. In some forms, the crosslinker is a bivalent, homofunctional linker, such as N,N′-methylene-bisacrylamide. In some forms, monomers used to form the macromolecule (e.g. 3-acryloylamino-propyl)-(2-carboxyethyl)-dimethyl ammonium) are polymerized in the presence of a crosslinker (e.g. N,N′-methylene-bisacrylamide) (as detailed in the examples), upon which polymerization and crosslinking of the polymers occur to produce hydrogels.

### (b) Self-Assembly

In some forms, the nanoparticles are formed by self-assembly of a mixture of polymers containing amphiphilic block copolymers in an aqueous solution. In an aqueous environment, the amphiphilic copolymers can spontaneously self-assemble to form nanoparticles with a hydrophobic core and a hydrophilic outer shell.

### (c) Solvent Diffusion/Displacement

In this method, water-soluble or water-miscible organic solvents are used to dissolve the polymer and form emulsion upon mixing with the aqueous phase. The quick diffusion of the organic solvent into water leads to the formation of nanoparticles immediately after the mixing.

### (d) Solvent Evaporation

In this method the polymer is dissolved in a volatile organic solvent. The drug (either soluble or dispersed as fine particles) is added to the solution, and the mixture is suspended in an aqueous solution that contains a surface active agent such as poly(vinyl alcohol). The resulting emulsion is stirred until most of the organic solvent evaporated, leaving solid nanoparticles. The resulting nanoparticles are washed with water and dried overnight in a lyophilizer. Nanoparticles with different sizes and morphologies can be obtained by this method.

### (e) Solvent Removal

In this method, the drug is dispersed or dissolved in a solution of the selected polymer in a volatile organic solvent. This mixture is suspended by stirring in an organic oil (such as silicon oil) to form an emulsion. Unlike solvent evaporation, this method can be used to make nanoparticles from polymers with high melting points and different molecular weights. The external morphology of spheres produced with this technique is highly dependent on the type of polymer used.

### (f) Spray-Drying

In this method, the polymer is dissolved in organic solvent. A known amount of the active drug is suspended (insoluble drugs) or co-dissolved (soluble drugs) in the polymer solution. The solution or the dispersion is then spray-dried.

### (g) Phase Inversion

Nanospheres can be formed from polymers using a phase inversion method wherein a polymer is dissolved in a “good” solvent, fine particles of a substance to be incorporated, such as a drug, are mixed or dissolved in the polymer solution, and the mixture is poured into a strong non solvent for the polymer, to spontaneously produce, under favorable conditions, polymeric microspheres, wherein the polymer is either coated with the particles or the particles are dispersed in the polymer. The method can be used to produce nanoparticles in a wide range of sizes, including, for example, about 100 nanometers to about 10 microns. Substances which can be incorporated include, for example, imaging agents such as fluorescent dyes, or biologically active molecules such as proteins or nucleic acids. In the process, the polymer is dissolved in an organic solvent and then contacted with a non-solvent, which causes phase inversion of the dissolved polymer to form small spherical particles, with a narrow size distribution optionally incorporating an antigen or other substance.

### (h) Microfluidics

Methods of making nanoparticles using microfluidics are known in the art. Suitable methods include those described in U.S. Patent Application Publication No. 2010/0022680 A1 by Karnik, et al. In general, the microfluidic device comprises at least two channels that converge into a mixing apparatus. The channels are typically formed by lithography, etching, embossing, or molding of a polymeric surface. A source of fluid is attached to each channel, and the application of pressure to the source causes the flow of the fluid in the channel The pressure may be applied by a syringe, a pump, and/or gravity. The inlet streams of solutions with polymer, targeting moieties, lipids, drug, payload, etc. converge and mix, and the resulting mixture is combined with a polymer non-solvent solution to form the nanoparticles having the desired size and density of moieties on the surface. By varying the pressure and flow rate in the inlet channels and the nature and composition of the fluid sources nanoparticles can be produced having reproducible size and structure.

### (3) Cell Surface Molecule Containing Abiotic Functional Group

Macromolecules, nanoparticles, or both, in the layer adjacent to the surface of the cell are conjugated to a cell surface molecule that has been modified to contain an abiotic functional group, such as those described above. The approached employed to introduce the abiotic functional group, exploits the biosynthetic machinery of the cell. For example, a cell can be incubated with a monosaccharide that has been modified to contain an azide group under conditions in which the monosaccharide is internalized into the cell. Following internalization, the monosaccharide can be incorporated into a carbohydrate via the cells carbohydrate biosynthetic machinery. The carbohydrate can then be transferred to a protein via post-translation modification to form a glycoprotein, or to a lipid to form a glycolipid. The glycoprotein or glycolipid can then be expressed on the extracellular surface of the cell, thereby display the abiotic functional group. Methods of introducing abiotic functional groups into cell surface molecules are described in Saxon and Bertozzi, Science 2000, 287, 2007-2010, and Stephan, et al., Nano Today 2011, 6(3).

### (4) Surface-Modified Cell

Following expression of an abiotic functional group on the surface of the cell, the abiotic functional group can be used to conjugate nanoparticles, macromolecules, or both, to form a first layer the surface of the cell, preferably using another functional group on the nanoparticles, macromolecules, or both. In some forms, the abiotic functional group on the surface of the cell is an azide. Preferably, the functional group on the nanoparticles, macromolecules, or both is an abiotic functional group. In some forms, the abiotic functional group on the nanoparticles, macromolecules, or both, is an alkyne, such as a cyclooctyne (dibenzocyclooctyne). Preferably, the conjugation is via covalent linkage containing a substituted triazole. Successive reactions can be performed to apply additional layers containing macromolecules, nanoparticles, or both, through a layer-by-layer conjugation scheme. The layer-by-layer conjugation scheme can be performed by alternating between azide- and alkyne-containing macromolecules or nanoparticles in successive conjugations. Preferably, the conjugation scheme is via covalent linkage.

### IV. Methods of Using

The surface-modified cell described herein can be used in cell-based therapy applications where improved performance (such as stable conformal coating, reduce foreign body response, increase cell viability) as compared to other cell-based therapy applications, are useful or preferred. These include, but are not limited to, inhibiting the proliferation of tumor cells in cancer patients, controlling blood sugar levels in diabetic patients, improving tissues in cardiovascular disease, and controlling obesity.

Methods of use typically involve administering to a host, in need thereof, a composition containing an effective amount of the surface-modified cells to inhibit progression of a disease.

The methods, compounds, and compositions herein described are further illustrated in the following examples, which are provided by way of illustration and are not intended to be limiting. It will be appreciated that variations in proportions and alternatives in elements of the components shown will be apparent to those skilled in the art and are within the scope of disclosed forms. All parts or amounts, unless otherwise specified, are by weight.

### EXAMPLES

### Example 1

### Conformal Coating of Pancreatic Islet Cells Materials and methods

Rat Pancreatic islets were isolated as previously reported (Veiseh, et al. Nat. Mater. 2015, 14, 643-651). Azido-sugars were purchased from Thermo Fisher Scientific. Span-80, N,N′ -Methylene-bisacrylamide, 1-Ethyl-3-(3-dimethylaminenopropyl)-1 carbodiimide hydrochloride (EDC), and N-Hydroxysulfosuccinimide (sulfo-NHS) were purchased from Sigma Aldrich. Amine-PEG4-DBCO, Amine-PEG4-Azide, Cy3-Azide and Cy5-DBCO were purchased from Click Chemistry. Zeba Resin Desalting (40K MWCO) columns were purchased from Thermo-Scientific.

(i) Synthesis of carboxybetaine acrylamide ((3-acryloylamino-propyl)-(2-carboxyethyl)-dimethyl ammonium; CBAA) monomer 1.6 g of N-[3-(dimethylamino) propyl] acrylamide (DMAPA, 98%, TCI America, OR) was mixed with with 0.99 g of β-propiolactone (90%, Sigma-Aldrich, WI) in 50 mL of anhydrous acetone at 0° C. for 2 h under nitrogen protection. The product appeared as a white precipitate, was washed with anhydrous ether, dried in vacuum, and stored at 4° C. Characterization: ¹H NMR (Bruker 500 MHz, DMSO-d6): 8.61 (t, 1H, N—H), 6.28 (t, 1H, CHH═CH), 6.13 (t, 1H, CHH═CH), 5.61 (t, 1H, CHH═CH), 3.44 (t, 2H, N—CH₂—CH₂—COO), 3.21 (m, 4H, NH—CH₂—CH₂—CH₂), 2.97 (s, 6H, N—(CH₃)₂), 2.25 (t, 2H, CH₂—COO), 1.87 (t, 2H, NH—CH₂—CH₂—CH₂).

### (ii) Synthesis of Hydrogels

CBAA hydrogels were synthesized in an inverse micro-emulsion polymerization using different quantities of reagents. For the organic solution, 0.4 g SPAN®-80 and 4 mg of V-70 were dissolved into 10 mL of hexane over ice. For the aqueous solution, 57 mg of the CBAA monomer and 2 mg of N,N′-methylene-bisacrylamide crosslinker were dissolved into 1 mL of DI water. The aqueous and organic solutions were combined in a 50 mL flask with vigorous stirring over ice. Sonication was used at 10° C. to create a microemulsion. The solution was purged with nitrogen gas for 15 minutes on ice and placed in a silicon oil bath at 40° C. for 2 hours over constant stirring throughout the polymerization. Following the reaction, the solution was washed 6 times with 25 mL of tetrahydrofuran (THF) at 5,000 rpm for 5 minutes. The hydrogels were resuspended in DI water with sonication. A dynamic light scattering (DLS) particle sizer (Nano ZS, Zetasizer Nano Malvern) characterized the hydrodynamic size of the hydrogels.

(iii) Functionalization of Hydrogels

The surfaces of the nanogels produced above were functionalized to contain an alkyne or an azide functional group.

200 mg of hydrogels were dispersed into 4.0 mL of 6.0 pH 0.1 M

MES buffer with 304 mg of EDC and 45 mg of NHS. This reaction proceeded for 1 hour on an orbital shaker and the solution was purified in a Zeba Resin column equilibrated with 8.0 pH 40 mM sodium bicarbonate buffer. The solution was split into two components. 20 mg of amine-PEG4-DBCO was added to one aliquot and 20 mg of amine-PEG4-azide was added to the other. These reactions proceeded for 3 hours on an orbital shaker and both solutions were purified in Zeba Resin columns equilibrated with 7.4 pH PBS. The functionalized nanoparticles were characterized with DLS particle sizer (Nano ZS, Zetasizer Nano Malvern) to determine their hydrodynamic size and stability throughout the functionalization process. Particle size was measured for seven days to verify the stability of the nanogels in PBS.

To prepare fluorescently labeled particles for imaging, 15 μg of Cy3-azide was added to the DBCO functionalized particles and 15 μg of Cy5-DBCO was added to the azide functionalized particles. These reactions proceeded for 3 hours on an orbital shaker in the absence of light and the solutions were purified in Zeba Resin columns equilibrated with 7.4 pH PBS.

### (iv) Expression of Azide Functional Groups on Cell Surface

GlcNAz, ManNAz and GalNAz Reagents are azido sugars that provide a highly specific approach for creating azide functional groups on cell surfaces through chemoselective ligation. Reactions using the azido sugars occur between a phosphine and an azide via a Staudinger reaction to produce an aza-ylide intermediate that is trapped to form a stable, covalent amide bond.

N-linked glycosylation is a modification of asparagine amines, whereas O-linked glycosylation occurs through the hydroxyl of serine and threonine residues. The azido sugars are bioorthogonal substitutes for endogenous amino sugars. ManNAz is converted by cells to an azido sialic acid derivative that is used for N-linked glycosylation of cell surface proteins. GlcNAz and GalNAz are predominantly used to label the O-linked glycosylation (O-GlcNAc and O-GalNAc).

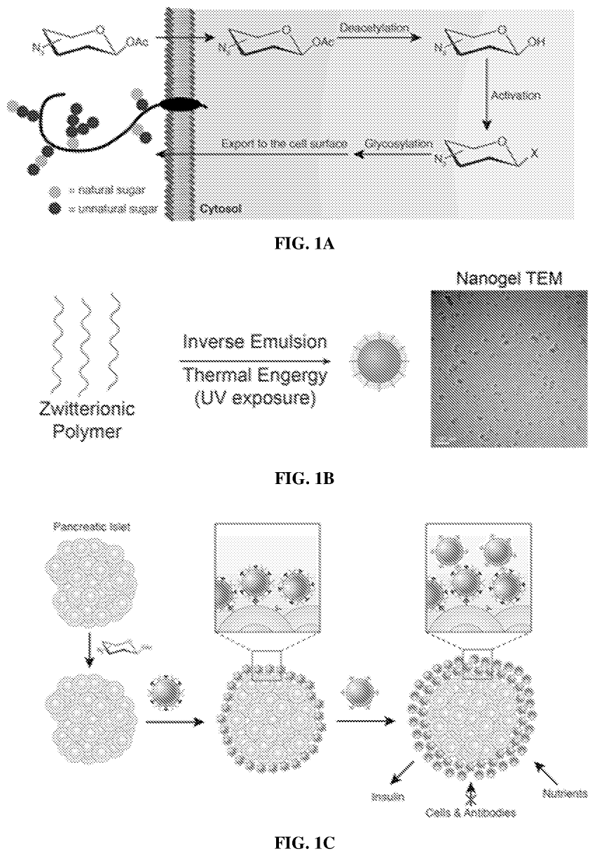

Rat pancreatic islet cells were induced to express azide groups on their extracellular surface, by incubating the cells with acetylated azido-sugars, FIG. 1A. 10 mM of reagent (azido-sugar) were prepared by dissolving 5 mg of the azido-sugar reagent in 1.16 mL of DMSO. The 10-mM solution of azido-sugars was added to the cell culture media for a final concentration of 40 μM (1:250 dilution) and incubated at 37° C. for 48 hours. The modified sugars undergo a Staudinger reaction to produce stable cell-surface adducts (Saxon and Bertozzi, Science 2000, 287, 2007-2010). The cells were incubated for at least 72 hours in an incubator to incorporate azido sugars into glycoproteins.

Confocal fluorescence imaging was used to verify that cells do express azide on their surface. To verify that cell surfaces where indeed modified to present azide functional groups, the cell surfaces were tested by reacting cell surfaces with DyLight 488-Phosphine. Fluorescence in confocal images was used to confirm successful expression of azides on cell surfaces.

These cell surface azide groups were used for covalent reactions on the surface of the cell.

(v) Reacting Azide Functional Group with Cyclooctyne to Apply Conformal Coating

Next, nanogels containing zwitterionic polymers, to which cyclooctynes have been covalently coupled (functionalized), were reacted with the azide groups forming a conformal coating around cell clusters. Briefly, following the modification of the cell surface, the nanogels with DBCO functionality were introduced to the cell media and incubated for 2 hours under constant shaking at 37° C. An additional layer of nanogels was added by reacting the nanogels of the first layer containing cyclooctynes with nanogels containing azide functional groups. Briefly, after thorough washing with PBS, the second nanoparticles with azide functionality were added to the cells in fresh media to attach a final layer of nanoparticles. The cells are incubated for 2 hours at 37° C. under constant shaking and are washed with PBS to remove any free-floating nanoparticles. More layers of nanogels can be added through a layer-by-layer covalent scheme by alternating between cyclooctyne- and azide-containing nanogels.

Two groups of conformally coated rat islet cells were produced. The first and second groups contained one and two layers of fluorescently labeled zwitterionic nanogels. Each group was then cultured in vitro for one week.

### (vi) Assessing the Stability of the Conformal Coatings

After a one-week time period, the structural integrity of the coatings was assessed using confocal fluorescence microscopy.

(vii) Glucose Stimulated Insulin Secretion Assay

The glucose stimulated insulin secretion (GSIS) assay was performed to determine the viability of the coated islet cells. The cells were first equilibrated in KREBS Buffer with 0.1% bovine serum albumin (BSA) at 37° C. for one hour before being transferred to a low glucose solution of 2 mM glucose in KREBS buffer with 0.1% BSA. The cells were incubated in the low glucose solution for 30 minutes at 37° C. Following sample collection of the supernatant, the cells were washed with PBS and incubated in a high glucose solution of 20 mM glucose in KREBS with 0.1% BSA for one hour at 37° C. Samples were collected from the high glucose solution and an ELISA assay was used to quantify the insulin concentration of the low and high glucose samples. The remaining cells were lysed for DNA quantification with FluoReporter Blue Fluorometric dsDNA Quantification Kit (Life Technologies) and the insulin concentrations were normalized per cell number.

(viii) Immunostaining

Immunofluorescence imaging was used to visualize the conformal coating of cells. Coated cells were fixed using 4% paraformaldehyde at room temperature for one hour. Samples were washed with PBS and incubated for one hour in an immunostaining solution of DAPI (500 nM) and wheat germ agglutin AlexaFluor 488 (5 μg/mL). Following staining, samples were washed with PBS and suspended in 50% glycerol solution. The samples were transferred to glass bottom plates for imaging.

### Results

### (i) Expression of Azide Functional Groups on Cell Surface

Fluorescence in confocal images confirmed successful expression of azides on cell surfaces.

### (ii) Assessing the Stability of the Conformal Coatings

The images captured from the confocal fluorescence microscopy demonstrated that the conformal containing two layers displayed improved stability around the islets compared to the conformal coating containing one layer.

### Example 2

### Incorporating Magnetic Resonance Imaging (MRI) Contrast Agents

### Materials and Methods

### (i) Synthesis of Iron-Containing Hydrogels

Zwitterionic polymers were chosen for the encapsulation matrix because the dual charges on the polymer side chain increase the surface wetting of the material. This allows for the material to masquerade as water and avoid the natural autoimmune response of the body. The nanogels are synthesized through an inverse microemulsion polymerization scheme to create polymer beads of between 50 nm and 200 nm in diameter adapted from Zhang, et al., ACS Nano 2012, 6(8), 6681-6686. As in Example 1, the polymerization uses a carboxybetaine acrylamide (CBAA) monomer and a methylene-bisacrylamide crosslinker. The nanogels have an iron oxide core to incorporate magnetic properties within the nanogels.

An iron solution of 0.21 g iron (II) chloride and 0.36 g iron (III) chloride dissolved in 18 mL of de-oxygenated distilled water was used as the solvent for the aqueous solution. For the aqueous component, 57 mg of the CBAA monomer and 2 mg of N,N′-methylene-bisacrylamide crosslinker were dissolved into 1 mL of the previously prepared aqueous iron solution. 0.4 g Span-80 and 4 mg of V-70 was dissolved into 10 mL of hexane over ice for the organic solution. The aqueous and organic solutions were combined in a 50 mL flask with vigorous stirring over ice. Sonication was used at 10° C. to create a micro-emulsion. The solution was purged with nitrogen gas for 15 minutes on ice and placed in a silicon oil bath at 40° C. for 2 hours with constant stirring. Following the reaction, the solution was purged again with nitrogen gas. In order to precipitate iron ions into iron oxide (Fe₃O₄), 1.5 mL of deoxygenated ammonium hydroxide was added to the reaction mixture dropwise over constant accelerated stirring and nitrogen gas to prevent oxidation. To prevent aggregation of the iron particles, the solution was stirred under sonication for 10 minutes at room temperature. The solution was washed 6 times with 25 mL of tetrahydrofuran (THF) at 5,000 rpm for 5 minutes. The hydrogels were resuspended in DI water with sonication. A dynamic light scattering (DLS) particle sizer (Nano ZS, Zetasizer Nano Malvern) characterized the hydrodynamic size of the hydrogels.

The size and size distribution of the nanogels were verified through dynamic light scattering (DLS) techniques as well as cryoTEM imaging. The surfaces of the nanogels were further modified through carbodiimide crosslink chemistry in order to create two distinct sets of nanoparticles. One set of nanoparticles contained a dibenzocyclooctyne (DBCO) group and the other contained azide (N₃) functionalization. These functional groups were chosen, because they can participate in a click chemistry, which leads to fast reactions with high conversion. The inclusion of these functional groups on the nanoparticles was verified using nuclear magnetic resonance (NMR) spectroscopy.

### (ii) Iron Quantification

Iron uptake was quantified using a modified Ferrozine assay. First two buffers were prepared: (1) an iron releasing regent was made of 0.7 M hydrochloric acid with 2.25% potassium permanganate, and (2) a Ferrozine solution was made of 1 M ascorbic acid, 2.5 M ammonium acetate, 6.5 mM ferrozine, 0.135% neocuproine. The cells were then removed from media and washed with PBS to remove any unbounded iron-containing nanoparticles. The cells were lysed with 300 μL of 50 mM sodium hydroxide and vortexed for 30 seconds. The cells were neutralized with 300 μL of 10 mM hydrochloric acid. Next, the cells were quantified using Bio-Safe Coomassie Blue Reagent and measuring absorbance at 595 nm. To quantify the iron, 300 μL of iron-releasing reagent was add to 300 μL of cell lysate and incubated at 60° C. for two hours. The samples were allowed to cool to room temperature before 90 μL of ferrozine solution was added. The samples were incubated for 30 minutes at room temperature before the absorbance was measured at 562 nm. The measured absorbance was compared to a curve generated from iron standards.

(iii) Coating Islet Cell Clusters

To coat the islet cell clusters, the cell membranes of the islet cells were modified with azido sugars. These sugars were added to the cell media after the islet cells were harvested and isolated. The cells ingested these sugars and then expressed the azide functional group on their cell membrane, which allowed for forming covalent bonds to the cell surfaces. Verification that the azido sugars had been expressed on the surfaces of the cells was conducted through fluorescent immunosorbent staining. Then the two sets of nanoparticles were applied as successive layers on to the cell surfaces. The DBCO nanoparticles first reacted with the azide functionality expressed on the cell surface followed by the azide nanoparticles, which reacted with the alkyne bond in the DBCO nanoparticles. Without both layers of nanoparticles, the particles are internalized within the islet cell cluster and cannot protect and encapsulate the cells. Confocal microscopy was used to visualize the conformally coated islet clusters and verify that the coating was successful.

The autoimmune response of the body will compromise the transplants by invoking a fibrotic response. Fibrosis will not allow for the transport of materials to and from the islet cells.

### Results

MRI contrast agents containing magnetic nanoparticles were successfully integrated into the nanogels described above, and the nanogels covalently linked to the surfaces of rat pancreatic islet following the methods described in Example 1 above. Transmission electron microscopy images confirmed the presence of the magnetic nanoparticles within the nanogels. Further, cells labeled with the magnetic nanoparticles could be tracked using MRI. For example, the nanogels were visible near the right hind leg of the mouse.

### Example 3

### Conformal Coating of Lymphocytes (T and B Cells)

### Materials and Methods

T and B cells from isolated from human peripheral blood were coated using the conformal coating strategy described above. Azido-sugar reagents were prepared as stated above and added to the cell culture media for a final concentration of 40 μM. T and B cells were incubated between 48 hours and 72 hours for sufficient incorporation of azido sugars into glycoproteins. The azido groups were then reacted with cyclooctyne functionalized nanoparticles, forming covalent linkages over 2 hours with constant shaking at 37° C. T and B cells were then washed with PBS. Azide functionalized nanogels were then added with fresh media to the cells for 2 hours of incubation to complete the first layer. Additional layers can be added by alternating between addition of azide and cyclooctyne functionalized nanoparticles, forming additional completed layers.

### Results

Confocal fluorescence images confirmed the successful conformal coating of the surfaces of individual cells.

Unless defined otherwise, all technical and scientific terms used herein have the same meanings as commonly understood by one of skill in the art to which the disclosed invention belongs. Publications cited herein and the materials for which they are cited are specifically incorporated by reference.

1. A surface-modified cell, comprising:
a cell
and a conformal coating on the extracellular surface of the cell, wherein the conformal coating comprises two or more layers comprising particles, macromolecules, or both; and
a covalent linkage between
(i) the particles, macromolecules, or both, in a first layer and a molecule on the surface of the cell,
(ii) the particles, macromolecules, or both, in one layer and particles, macromolecules, or both, in an adjacent layer of the conformal coating, or
(iii) a combination of (i) and (ii).

2. The surface-modified cell of claim 1, wherein the conformal coating partially or completely covers the surface of the cell.

3. The surface-modified cell of claim 1, wherein the conformal coating is more stable compared to a corresponding conformal coating having one layer on a corresponding cell, as determined by observing the structural integrity of the conformal coating using confocal fluorescence microscopy.

4. The surface-modified cell of claim 1, wherein the number of layers in the conformal coating controls the residence time of the surface-modified cell, as determined by confocal fluorescence imaging.

5. The surface-modified cell of claim 1, comprising a covalent linkage between the particles, macromolecules, or both, in the first layer and the molecule on the surface of the cell.

6. The surface-modified cell of claim 1, comprising a covalent linkage between the particles, macromolecules, or both, in the first layer and the particles, macromolecules, or both, in an adjacent layer.

7. The surface-modified cell of claim 1, comprising three or more layers, and a covalent linkage between the particles, macromolecules, or both, in adjacent layers.

8. The surface-modified cell of claim 1, wherein the layers comprise particles.

9. The surface-modified cell of claim 1, wherein the layers comprise macromolecules.

10. The surface-modified cell of claim 1, wherein the covalent linkage comprises a substituted triazole, an amide, a carbamate, oxime ether, hydrazone, thio-ether, a carbonyl, imine, sulfonamide, azo, dialkyl dialkoxysilane, diaryl dialkoxysilane, orthoester, acetal, aconityl, β-thiopropionate, phosphoramidate, trityl, vinyl ether, polyketal, or a combination thereof.

11. The surface-modified cell of claim 1, wherein the covalent linkage is between the particles, macromolecules, or both, in the first layer and the molecule on the surface of the cell.

12. The surface-modified cell of claim 11, wherein the molecule on the surface of the cell is a biomolecule selected from the group consisting of proteins, glycoproteins, lipids, glycolipids, and combinations thereof.

13. The surface-modified cell of claim 1, wherein the particles have a diameter between about 1 nm and about 5 μm.

14. The surface-modified cell of claim 1, wherein the conformal coating has a thickness between about 1 nm and about 5 μm.

15. The surface-modified cell of claim 1, wherein the particles, macromolecules, or both, comprise diagnostic, therapeutic, prophylactic, and/or targeting agents.

16. The surface-modified cell of claim 15, wherein the diagnostic agent is an iron oxide nanoparticle.

17. The surface-modified cell of claim 1, wherein the particles, macromolecules, or both, comprise a polymer comprising a backbone formed from a polymer selected from the group consisting of poly(acrylate), poly(methacrylate), poly(acrylamide), poly(methacrylamide), poly(vinyl alcohol), poly(ethylene vinyl acetate), poly(vinyl acetate), poly(olefin), poly(ester), poly(hydroxyalkanoates), poly(anhydride), poly (orthoester), polyamide, polyamine, polyether, polyazine, poly(carbonate), polyetheretherketone (PEEK), poly(amino acids), polyimide, polyketal, poly(ketone), polyphosphazine, alginates, polysaccharide, polysiloxane, polysulfone, polyurea, poly(urethane), poly(alkylene oxide), blends, and copolymers thereof.

18. The surface-modified cell of claim 17, wherein the polymer comprises a zwitterionic polymer, a chemically modified alginate, blends, or copolymers thereof,
wherein the chemically modified alginate comprises a covalently modified monomer having the structure: 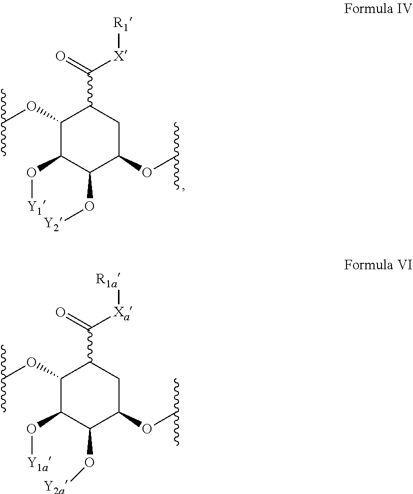
or a combination thereof,
X′ is oxygen, sulfur, or NR₄′; Xₐ′ is oxygen, sulfur, or NR₄ₐ′;
R₁′ and R₁ₐ′ are, independently in the one or more modified monomers, hydrogen, alkyl, substituted alkyl, alkenyl, substituted alkenyl, alkynyl, substituted alkynyl, phenyl, substituted phenyl, aryl, substituted aryl, heteroaryl, substituted heteroaryl, alkoxy, substituted alkoxy, phenoxy, substituted phenoxy, aroxy, substituted aroxy, alkylthio, substituted alkylthio, phenylthio, substituted phenylthio, arylthio, substituted arylthio, carbonyl, substituted carbonyl, carboxyl, substituted carboxyl, amino, substituted amino, amido, substituted amido, sulfonyl, substituted sulfonyl, sulfonic acid, phosphoryl, substituted phosphoryl, phosphonyl, substituted phosphonyl, polyaryl, substituted polyaryl, C₃-C₂₀ cyclic, substituted C₃-C₂₀ cyclic, heterocyclic, substituted heterocyclic, amino acid, poly(ethylene glycol), peptide, polypeptide group;
Y₁′ and Y₂′ are independently hydrogen, —PO(OR₅′)₂; Y₁ₐ′ and Y₂ₐ′ are independently hydrogen, —PO(OR₅ₐ′)₂; or (i) Y₂′ is absent, and Y₁′, together with the two oxygen atoms to which Y₁′ and Y₂′ are attached form a cyclic structure as shown in Formula V 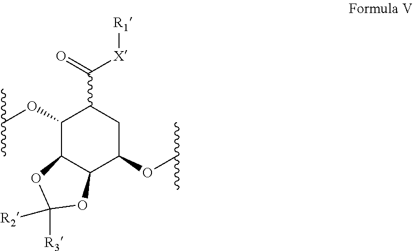
(ii) Y₂ₐ′ is absent, and Y₁ₐ′, together with the two oxygen atoms to which Y₁ₐ′ and Y₂ₐ′ are attached form a cyclic structure as shown in Formula VII 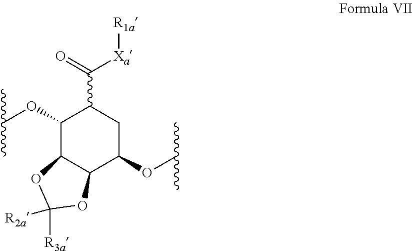
wherein
R₂′, R₃′, R₂ₐ′, and R₃ₐ′ are, independently, hydrogen, alkyl, substituted alkyl, alkenyl, substituted alkenyl, alkynyl, substituted alkynyl, phenyl, substituted phenyl, aryl, substituted aryl, heteroaryl, substituted heteroaryl, alkoxy, substituted alkoxy, phenoxy, substituted phenoxy, aroxy, substituted aroxy, alkylthio, substituted alkylthio, phenylthio, substituted phenylthio, arylthio, substituted arylthio, carbonyl, substituted carbonyl, carboxyl, substituted carboxyl, amino, substituted amino, amido, substituted amido, polyaryl, substituted polyaryl, C₃-C₂₀ cyclic, substituted C₃-C₂₀ cyclic, heterocyclic, substituted heterocyclic, amino acid, poly(ethylene glycol), peptide, or polypeptide group; or R₂ₐ′ and R₃ₐ′, together with the carbon atom to which they are attached, form a 3- to 8-membered unsubstituted or substituted carbocyclic or heterocyclic ring; and
R₄′, R₅′, R₄ₐ′ and R₅ₐ′ are, independently, hydrogen, alkyl, substituted alkyl, alkenyl, substituted alkenyl, alkynyl, substituted alkynyl, phenyl, substituted phenyl, aryl, substituted aryl, heteroaryl, substituted heteroaryl, alkoxy, substituted alkoxy, phenoxy, substituted phenoxy, aroxy, substituted aroxy, alkylthio, substituted alkylthio, phenylthio, substituted phenylthio, arylthio, substituted arylthio, carbonyl, substituted carbonyl, carboxyl, substituted carboxyl, amino, substituted amino, amido, substituted amido, polyaryl, substituted polyaryl, C₃-C₂₀ cyclic, substituted C₃-C₂₀ cyclic, heterocyclic, substituted heterocyclic, amino acid, poly(ethylene glycol), peptide, or polypeptide group;
with the proviso that at least one of R₁′, Y₁′, Y₂′, R₂′ and R₃′ is Formula I:
d-R₁—Y, Formula I
wherein:
R₁ is alkyl, substituted alkyl, alkenyl, substituted alkenyl, alkynyl, substituted alkynyl, aryl, substituted aryl, heteroaryl, substituted heteroaryl, alkoxy, substituted alkoxy, aroxy, substituted aroxy, alkylthio, substituted alkylthio, arylthio, substituted arylthio, carbonyl, substituted carbonyl, carboxyl, substituted carboxyl, amino, substituted amino, amido, substituted amido, sulfonyl, substituted sulfonyl, sulfamoyl, substituted sulfamoyl, phosphonyl, substituted phosphonyl, polyaryl, substituted polyaryl, C₃-C₂₀ cyclic, substituted C₃-C₂₀ cyclic, heterocyclic, substituted heterocyclic, amino acid, poly(ethylene glycol), poly(lactic-co-glycolic acid), peptide, or polypeptide group; and
Y is the covalent linkage selected from substituted triazoles, amides, carbamates, oxime ethers, hydrazones, thio-ethers, carbonyls, imines, sulfonamides, azo groups, dialkyl dialkoxysilanes, diaryl dialkoxysilanes, orthoesters, acetals, aconityls, β-thiopropionates, phosphoramidates, trityls, vinyl ethers, polyketals, or a combination thereof.

19. The surface-modified cell of claim 1, wherein the particles comprise nanoparticles.

20. The surface-modified cell of claim 19, wherein the nanoparticle are nanogels.

21. The surface-modified cell of claim 1, wherein the cell is selected from the group consisting of secretory cells, immune cells, metabolic cells, structural cells, aggregates thereof, and combinations thereof.

22. The surface-modified cell of claim 1, wherein the cell is a secretory cell.

23. The surface-modified cell of claim 1, wherein the cell is an immune cell.

24. A method of making a surface-modified cell comprising:
(i) incubating a cell with a building block of a biomolecule containing a first abiotic functional group, under conditions in which the building block is internalized by the cell and expressed on the extracellular surface of the cell;
(ii) conjugating the abiotic functional group with a second functional group conjugated to particles, macromolecules, or both, to form a layer around the cell; and
(iii) conjugating additional particles, macromolecules, or both having a third functional to the particles and/or macromolecules in the layer of (ii) to form another layer over the layer of (ii).

25. The method of claim 24, further comprising (iv) conjugating additional particles, macromolecules, or both, having the first, second, and/or third abiotic functional group, one or more times successively, to form one or more additional layers over the layer of step (iii).

26. The method of claim 24, wherein the conjugating step in (iii) is via covalent linkage.

27. The method of claim 24, wherein the first abiotic functional group is selected from the group consisting of azides, alkynes, alkenes, triarylphosphines, aminooxys, carbonyls, hydrazides, sulfonyl chlorides, maleimides, aziridines, —CN, acryloyls, acrylamides, vinyl sulfones, cyanates, thiocyanates, isocyanates, isothiocyanates, alkoxysilanes, vinyl silanes, acetohydrazides, acyl azides, acyl halides, epoxides, glycidyls, carbodiimides, and combinations thereof.

28. The method of claim 24, wherein the second and/or third functional group is selected from azides, alkynes, alkenes, triarylphosphines, aminooxys, carbonyls, hydrazides, sulfonyl chlorides, maleimides, aziridines, —CN, acryloyls, acrylamides, vinyl sulfones, cyanates, thiocyanates, isocyanates, isothiocyanates, alkoxysilanes, vinyl silanes, acetohydrazides, acyl azides, acyl halides, epoxides, glycidyls, carbodiimides, thiols, amines, or a combination thereof.

29. The method of claim 24, wherein the building block is a building block of a biomolecule selected from the group consisting of proteins, glycoproteins, lipids, glycolipids, and combinations thereof.

30. The method of claim 24, wherein the second and/or third functional group is conjugated to a material from which the particles are formed before or after particle formation.

31. The method of claim 24, wherein the particles, macromolecules, or both, comprise a polymer comprising a backbone formed from a poly(acrylate), poly(methacrylate), poly(acrylamide), poly(methacrylamide), poly(vinyl alcohol), poly(ethylene vinyl acetate), poly(vinyl acetate), poly(olefin), poly(ester), poly(hydroxyalkanoates), poly(anhydride), poly (orthoester), polyamide, polyamine, polyether, polyazine, poly(carbonate), polyetheretherketone (PEEK), poly(amino acids), polyimide, polyketal, poly(ketone), polyphosphazine, alginates, polysaccharide, polysiloxane, polysulfone, polyurea, poly(urethane), poly(alkylene oxide), blends, or copolymers thereof.

32. The method of claim 31, wherein the second and/or third functional group is conjugated to the backbone of the polymer.

33. The method of claim 24, wherein the building block containing the abiotic functional group is selected from the group consisting of amino acids, glycans, glycolipids, lipids, and a combination thereof.

34. The method of claim 24, wherein the first abiotic functional group is an azide.

35. The method of claim 24, wherein the second and/or third functional group is selected from alkynes, azides, or a combination thereof.

36. The method of claim 27, wherein at least one of the alkynes is a cyclooctyne.

37. The method of claim 24, wherein the particles are nanoparticles.

38. The method of claim 37, wherein the nanoparticles are nanogels.
